(19) United States
(12) Patent Application Publication
(10) Pub. No.: US 2026/0207177 A1
(43) Pub. Date: Jul. 23, 2026

(54) SYSTEMS AND METHODS FOR HIGH RESOLUTION ULTRASOUND IMAGING ARTIFACT REDUCTION

(71) Applicant: Ulthera, Inc., Raleigh, NC (US)

(72) Inventors: Stephen John Hsu, Mesa, AZ (US); Charles D. Emery, Gilbert, AZ (US); Douglas J. Halbert, Gilbert, AZ (US)

(21) Appl. No.: 19/140,689

(22) PCT Filed: Nov. 3, 2023
(86) PCT No.: PCT/US2023/078607

§ 371 Date: Jun. 18, 2025

Related U.S. Application Data
(60) Provisional application No. 63/476,319, filed on Dec. 20, 2022.

(51) Int. Cl. A61B 8/00; A61B 8/08

(52) U.S. Cl. CPC A61B 8/5276; A61B 8/0858; A61B 8/4461

(57) ABSTRACT

Enhancements of resolution of high speed ultrasound imaging of tissue in association with aesthetic and/or cosmetic treatments of skin and/or tissue near the skin. In one embodiment, high resolution ultrasound imaging uses dynamic focal zone blending to reduce an appearance of acoustic window multipath echo artifacts. In one embodiment, high resolution ultrasound imaging uses an offset between a first imaging frame in a first direction and a second imaging frame in a second direction to reduce a temporal motion artifact. In some embodiments, the imaging system is used with an aesthetic and/or cosmetic skin treatment.

### CROSS-REFERENCE TO RELATED APPLICATIONS

This application claims the benefit of priority from U.S. Provisional Application No. 63/476,319 filed Dec. 20, 2022, which is incorporated in its entirety by reference, herein. Any and all priority claims identified in the Application Data Sheet, or any correction thereto, are hereby incorporated by reference under 37 CFR 1.57.

### BACKGROUND

### Field

Several embodiments of the invention relate to enhancements of high resolution of high speed movement of ultrasound imaging of tissue in association with aesthetic and/or cosmetic treatments of skin and/or tissue near the skin. In one embodiment, high resolution ultrasound imaging uses dynamic focal zone blending to reduce an appearance of acoustic window multipath echo artifacts brought about from high frame rate and/or high speed movement of an ultrasound imaging transducer. In one embodiment, high resolution ultrasound imaging uses an offset between a first imaging frame in a first direction and a second imaging frame in a second direction to reduce a temporal motion artifacts.

### Description of the Related Art

Conventional ultrasound imaging generally uses a single focal zone with a stationary ultrasound imaging transducer.

### SUMMARY

There is a need for improved resolution for offset ultrasound imaging using multiple focal zones at high speeds to quickly, efficiently, and accurately image tissue for aesthetic and/or cosmetic treatments of skin and/or tissue underlying the skin. In various embodiments, an ultrasound system is configured for imaging to visualize tissue (e.g., epidermal, dermal and/or subdermal layers of tissue). In various embodiments, an ultrasound system is configured for imaging to visualize tissue (e.g., epidermal, dermal and/or subdermal layers of tissue) to confirm appropriate depth of an associated cosmetic or medical treatment such as to avoid certain tissues (e.g., nerve, bone).

In various embodiments, systems and methods for ultrasound imaging of tissue are adapted for and/or configured to use one or more focal zones in the tissue for imaging. In one embodiment, one single focal zone is used for imaging. In various embodiments, two, three, four, or more focal zones are used for imaging. In various embodiments, an ultrasound transducer for imaging is placed directly in contact through acoustic coupling to a tissue such as a skin surface for imaging the one or more focal zones under the skin surface. In various embodiments, an ultrasound transducer for imaging has an offset gap between the imaging transducer and a portion of a housing (such as at a window, such as a PEEK window) in an ultrasound probe, whereby the portion of the housing is placed in contact through acoustic coupling to a tissue such as a skin surface for imaging the one or more focal zones under the skin surface. In some embodiments, an ultrasound transducer for imaging has an offset gap between the imaging transducer and a portion of a housing that uses two or more (e.g., 2, 3, 4, 5, 6, or more) focal zones that can produce multipath artifacts from acoustic ultrasound energy that bounces between the imaging transducer and (i) the acoustic window and/or (ii) the region being imaged. These artifacts may obscure the clarity of the imaging. In various embodiments described herein, systems and methods reduce and/or eliminate such artifacts.

In various embodiments, ultrasound imaging is used to visualize a tissue region and/or anatomy. In one embodiment, ultrasound imaging is used to confirm sufficient acoustic coupling to a tissue region for improving imaging correlation between movement of the ultrasound imaging transducer in a first and second direction when forming images.

In various embodiments, ultrasound imaging is used in conjunction with a cosmetic treatment or a medical treatment in order to visualize, plan and/or monitor the cosmetic or medical treatment. In one embodiment, ultrasound imaging is used in conjunction with an application of energy to a tissue. In one embodiment, ultrasound imaging is used in conjunction with an application of ultrasound therapy to a tissue. In one embodiment, ultrasound imaging is used in conjunction with an application of a dermal filler to a tissue. In one embodiment, ultrasound imaging is used in conjunction with an application of a drug or a compound to a tissue. In one embodiment, ultrasound imaging is used in conjunction with an application of a botulinum toxin to a tissue.

In several embodiments, provided are systems and methods that successfully achieve an aesthetic effect using targeted and precise ultrasound to cause a visible and effective cosmetic result via a thermal pathway by splitting an ultrasound therapy beam to two, three, four, or more simultaneous focal zones for performing various treatment and/or imaging procedures. In various embodiments, an ultrasound system is configured for focusing ultrasound to produce localized, mechanical motion within tissues and cells for the purpose of producing either localized heating for tissue coagulation or for mechanical cellular membrane disruption intended for non-invasive aesthetic use. In various embodiments, an ultrasound system is configured for lifting a brow (e.g., an eyebrow). In various embodiments, an ultrasound system is configured for lifting lift lax tissue, such as submental (beneath the chin) and neck tissue. In various embodiments, an ultrasound system is configured for improving lines and wrinkles of the décolleté. In various embodiments, an ultrasound system is configured for reducing fat. In various embodiments, an ultrasound system is configured for reducing the appearance of cellulite.

In several embodiments disclosed herein, non-invasive ultrasound systems are adapted to be used in achieving one or more of the following beneficial aesthetic and/or cosmetic improvement effects: a face lift, a brow lift, a chin lift, an eye treatment (e.g., malar bags, treat infraorbital laxity), a wrinkle reduction, fat reduction (e.g., treatment of adipose and/or cellulite), cellulite (which may be called gynoid lipodystrophy) treatment (e.g., dimple or non-dimple type female gynoid lipodystrophy), décolletage improvement (e.g., upper chest), a buttock lift (e.g., buttock tightening), skin tightening (for example, treating laxity to cause tightening on the face or body, such as the face, neck, chest, arms, thighs, abdomen, buttocks. etc.), a scar reduction, a burn treatment, a tattoo removal, a vein removal, a vein reduction, a treatment on a sweat gland, a treatment of hyperhidrosis, a sun spot removal, an acne treatment, a pimple reduction.

Several embodiments are particularly advantageous because they include one, several or all of the following benefits: faster imaging time, (ii) higher imaging resolution, (iii) removal of obscuring artifacts from imaging, (iv) clear imaging from a moving imaging transducer, (v) more efficient imaging, and/or (vi) improved imaging to assist in associated treatment or therapy.

In several embodiments, an ultrasound imaging system configured for reducing imaging artifacts, including: an ultrasonic probe including: an ultrasound imaging transducer adapted for imaging a tissue region, a housing including an acoustic window, a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance, an acoustic coupling medium within the housing configured to acoustically couple the ultrasound imaging transducer to the acoustic window, a motion mechanism for moving the ultrasound imaging transducer in a first direction and a second direction, wherein the ultrasound imaging transducer images with a focal zone sequence order (f₁, . . . , f_{N}), where N>2 when travelling in the first direction, wherein the ultrasound imaging transducer images with a second focal zone sequence order (f₁, . . . , f_{N}) when travelling in the second direction; and a control module coupled to the ultrasonic probe for controlling the ultrasound imaging transducer, wherein the control module is configured to reduce at least one multipath echo artifact via a dynamically set pulse repetition interval.

In one embodiment, wherein the dynamically set pulse repetition interval is further configured to: measure the first offset depth; calculate a first offset time based on the first offset depth; multiply the first offset time by an integer to determine a presence of the at least one multipath echo artifact; and select a pulse repetition interval configured to position the at least one multipath echo artifact outside of a displayed ultrasound image.

In several embodiments, An ultrasound imaging system configured for reducing imaging artifacts, including: an ultrasonic probe including: an ultrasound imaging transducer adapted for imaging a tissue region, a housing including an acoustic window, a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance, an acoustic coupling medium within the housing configured to acoustically couple the ultrasound imaging transducer to the acoustic window, a motion mechanism for moving the ultrasound imaging transducer in a first direction and a second direction, wherein the ultrasound imaging transducer images with a focal zone sequence order (f₁, . . . , f_{N}), where N>2 when travelling in the first direction, wherein the ultrasound imaging transducer images with a second focal zone sequence order (f₁, . . . ,f_{N}) when travelling in the second direction; and a control module coupled to the ultrasonic probe for controlling the ultrasound imaging transducer, wherein the control module is configured to reduce at least one multipath echo artifact via a dynamically set one or more focal zone blend points.

In one embodiment, the at least one dynamically set focal zone blend point is further configured to: measure the first offset depth; calculate a first offset time based on the first offset depth; multiply the first offset time by an integer to determine a presence of the at least one multipath echo artifact; and select at least one focal zone blend point configured to position the at least one multipath echo artifact outside of a displayed ultrasound image. In one embodiment, the dynamic offset distance varies based on a changing volume of the acoustic coupling medium, wherein the changing volume of the acoustic coupling medium is a result of evaporation or leaking of the acoustic coupling medium from the housing. In one embodiment, the dynamic offset distance varies based on a changing temperature of the acoustic coupling medium. In one embodiment, the dynamic offset distance varies based on a changing pressure of the acoustic coupling medium. In one embodiment, the dynamic offset distance varies with a speed of the motion mechanism in at least one of the first direction and the second direction. In one embodiment, the device further includes a therapy transducer configured to apply ultrasonic therapy to the tissue. In one embodiment, N=any one of 2, 3, or 4.

In several embodiments, An ultrasound imaging system configured for reducing imaging artifacts, including: an ultrasonic probe including: an ultrasound imaging transducer adapted for imaging a tissue region, a housing including an acoustic window, a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance, means for moving the ultrasound imaging transducer in a first direction and a second direction, and a control module coupled to the ultrasonic probe for controlling the ultrasound imaging transducer, wherein the control module is configured to reduce at least one multipath echo artifact via a dynamically set pulse repetition interval.

In several embodiments, An ultrasound imaging module configured for reducing imaging artifacts, including: an ultrasound imaging transducer adapted for imaging a tissue region, a housing including an acoustic window, a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance, means for moving the ultrasound imaging transducer in a first direction and a second direction, and a control module coupled to the ultrasonic probe for controlling the ultrasound imaging transducer, wherein the control module is configured to reduce at least one multipath echo artifact via a dynamically set pulse repetition interval.

In one embodiment, the at least one dynamically set focal zone blend points is further configured to: measure the first offset depth; calculate a first offset time based on the first offset depth; multiply the first offset time by an integer to determine a presence of the at least one multipath echo artifact; and select a at least one focal zone blend point configured to position the at least one multipath echo artifact outside of a displayed ultrasound image.

In several embodiments, An ultrasound imaging device configured for reducing imaging artifacts, including: an ultrasonic module including: an ultrasound imaging transducer adapted for imaging a tissue region, a housing including an acoustic window, a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance, means for moving the ultrasound imaging transducer in a first direction and a second direction, and a control module coupled to the ultrasonic probe for controlling the ultrasound imaging transducer, wherein the control module is configured to reduce at least one multipath echo artifact via a dynamically set pulse repetition interval.

In one embodiment, the dynamically set at least one focal zone blend point is further configured to: measure the first offset depth; calculate a first offset time based on the first offset depth; multiply the first offset time by an integer to determine a presence of the at least one multipath echo artifact; and select at least one focal zone blend point configured to position the at least one multipath echo artifact outside of a produced ultrasound image. In one embodiment, the dynamic offset distance varies based on a changing volume of the acoustic coupling medium, wherein the changing volume of the acoustic coupling medium is a result of evaporation or leaking of the acoustic coupling medium from the housing. In one embodiment, the dynamic offset distance varies based on a changing temperature of the acoustic coupling medium. In one embodiment, the dynamic offset distance varies based on a changing pressure of the acoustic coupling medium. In one embodiment, the dynamic offset distance varies with a speed of the mechanism in at least one of the first direction and the second direction. In one embodiment, the device further includes a therapy transducer configured to apply ultrasonic therapy to the tissue. In one embodiment, N=any one of 2, 3, or 4.

In several embodiments, a method of reducing multipath echo artifacts from an ultrasound image, including: providing an ultrasonic probe including: an ultrasound imaging transducer adapted for imaging a tissue region, a housing including an acoustic window, a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance, an acoustic coupling medium within the housing configured to acoustically couple the ultrasound imaging transducer to the acoustic window, a motion mechanism for moving the ultrasound imaging transducer in a first direction and a second direction, wherein the ultrasound imaging transducer images with a focal zone sequence order (f₁, . . . , f_{N}), where N>2 when travelling in the first direction, wherein the ultrasound imaging transducer images with a second focal zone sequence order (f₁, . . . , f_{N}) when travelling in the second direction; and measuring a first offset depth; calculating a first offset time based on the first offset depth; multiplying the first offset time by an integer to determine a presence of the at least one multipath echo artifact; and selecting a pulse repetition interval configured to position the at least one multipath echo artifact outside of a displayed ultrasound image.

In several embodiments, a method of reducing multipath echo artifacts from an ultrasound image, including: providing an ultrasonic probe including: an ultrasound imaging transducer adapted for imaging a tissue region, a housing including an acoustic window, a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance, an acoustic coupling medium within the housing configured to acoustically couple the ultrasound imaging transducer to the acoustic window, a motion mechanism for moving the ultrasound imaging transducer in a first direction and a second direction; calculating a first offset time based on the first offset depth; multiplying the first offset time by an integer to determine a presence of the at least one multipath echo artifact; and selecting at least one focal zone blend point configured to position the at least one multipath echo artifact outside of a displayed ultrasound image.

In one embodiment, the method further includes imaging a tissue, and displaying the tissue. In one embodiment, the method further includes imaging a tissue, and displaying the tissue, without treating the tissue. In one embodiment, the method further includes treating a tissue.

In several embodiments, A method of improving ultrasound imaging alignment by reducing spatial and temporal motion artifacts, including: providing an ultrasonic probe including: an ultrasound imaging transducer adapted for imaging a tissue region, a motion mechanism attached to the ultrasound imaging transducer; wherein the ultrasound imaging transducer images a first image with a focal zone sequence order (f₁, . . . , f_{N}), where N>2 when travelling in the first direction, wherein the ultrasound imaging transducer images a second image with a second focal zone sequence order (f₁, . . . , f_{N}) when travelling in the second direction; acquiring the first imaging frame; acquiring the second imaging frame; calculating offsets between the first imaging frame and the second imaging frame to determine a lateral misregistration; displaying the first imaging frame; and displaying the second imaging frame with the offsets applied to reduce a temporal motion artifact.

In one embodiment, the method further includes calculating an optimized image with at least one trigger offset; and applying the at least one trigger offset to subsequent image acquisitions, wherein the lateral misregistration is reduced due to application of the at least one trigger offset.

In several embodiments, A method of improving ultrasound imaging alignment by reducing spatial and temporal motion artifacts, including: providing an ultrasonic probe including: an ultrasound imaging transducer adapted for imaging a tissue region, a motion mechanism attached to the ultrasound imaging transducer; wherein the ultrasound imaging transducer images a first image with a focal zone sequence order (f₁, . . . , f_{N}), where N>2 when travelling in the first direction, wherein the ultrasound imaging transducer images a second image with a second focal zone sequence order (f₁, . . . , f_{N}) when travelling in the second direction; acquiring multiple (N>1) imaging frames; calculating a temporal average of at least two imaging frames; displaying the temporal average of the at least two imaging frames to reduce a temporal motion artifact.

In one embodiment, the method further includes calculating an optimized image with at least one trigger offset; and applying the at least one trigger offset to subsequent image acquisitions, wherein the averaging of N>1 successive imaging frames is enabled when spatial misregistration between the current and a previously acquired imaging frame is less than a predetermined threshold.

In several embodiments, a method of improving ultrasound imaging alignment by reducing spatial and temporal motion artifacts, including: providing an ultrasonic probe including: an ultrasound imaging transducer adapted for imaging a tissue region, a motion mechanism attached to the ultrasound imaging transducer; wherein the ultrasound imaging transducer images a first image with a focal zone sequence order (f₁, . . . , f_{N}), where N>2 when travelling in the first direction, wherein the ultrasound imaging transducer images a second image with a second focal zone sequence order (f₁, . . . , f_{N}) when travelling in the second direction; acquiring the first imaging frame; acquiring the second imaging frame; calculating offsets between the first imaging frame and the second imaging frame to determine lateral misregistrations; calculating a temporal average to the first imaging frame and the second imaging frame; displaying the temporal average of the first imaging frame and the offset to the second imaging frame to reduce a spatial and temporal motion artifact.

In one embodiment, the method further includes calculating an optimized image with at least one trigger offset; and applying the at least one trigger offset to the optimized image, wherein the lateral misregistration is reduced due to the application of the at least one trigger offset. In one embodiment, the method further includes imaging a tissue, and displaying the tissue. In one embodiment, the method further includes imaging a tissue, and displaying the tissue, without treating the tissue. In one embodiment, the method further includes treating a tissue.

In several embodiments, an ultrasound imaging system configured for reducing imaging misalignment, including: an ultrasonic probe including an ultrasound therapy transducer adapted to apply ultrasonic therapy to tissue, an ultrasound imaging transducer adapted for imaging the tissue, and a motion mechanism for moving the ultrasound imaging transducer in a first direction and a second direction, wherein the ultrasound imaging transducer is mechanically attached to the motion mechanism, wherein the first direction is opposite the second direction, wherein the ultrasound imaging transducer images with a focal zone sequence order (f₁, . . . , f_{N}), where N>1 when travelling in the first direction, wherein the ultrasound imaging transducer images with a second focal zone sequence order (f₁, . . . , f_{N}) when travelling in the second direction, wherein a spatial registration between the first direction imaging and the second direction imaging is improved by staggering a triggering location, wherein the ultrasound imaging system employs a directionally dependent focal zone sequencing (f₁, . . . , f_{N}) and (f₁, . . . , f_{N}) on consecutive A-lines; and a control module coupled to the ultrasonic probe for controlling the ultrasound imaging transducer.

In one embodiment, N=any one of the group consisting of: 2, 4, 6, and 8. In one embodiment, the first direction of motion of the transducer is any one or more of the group consisting of: linear, rotational, and curved; wherein the second direction is the reversed path of the first direction. In one embodiment, the ultrasonic treatment is at least one of: a face lift, a brow lift, a chin lift, an eye treatment, a wrinkle reduction, a décolletage improvement, a buttock lift, a scar reduction, a burn treatment, a skin tightening, a blood vessel reduction, a treatment of a sweat gland, a sun spot removal, a fat treatment, a cellulite treatment, a vaginal rejuvenation, an acne treatment, and abdominal laxity treatment.

The methods summarized above and set forth in further detail below describe certain actions taken by a practitioner; however, it should be understood that they can also include the instruction of those actions by another party. Thus, actions such as “moving an imaging transducer” include “instructing the movement of an imaging transducer.”

In some embodiments, the system comprises various features that are present as single features (as opposed to multiple features). Multiple features or components are provided in alternate embodiments. In various embodiments, the system comprises, consists essentially of, or consists of one, two, three, or more embodiments of any features or components disclosed herein. In some embodiments, a feature or component is not included and can be negatively disclaimed from a specific claim, such that the system is without such feature or component. In some embodiments, a method is performed without a step. In some embodiments, a system does not comprise a certain component. Further, areas of applicability will become apparent from the description provided herein. It should be understood that the description and specific examples are intended for purposes of illustration only and are not intended to limit the scope of the embodiments disclosed herein.

### BRIEF DESCRIPTION OF THE DRAWINGS

The drawings described herein are for illustration purposes only and are not intended to limit the scope of the present disclosure in any way. Embodiments of will become more fully understood from the detailed description and the accompanying drawings. Features from one drawing are applicable to other drawings in several embodiments.

FIG. 1A is a schematic illustration of an ultrasound system according to various embodiments.

FIG. 1B is a schematic illustration of an ultrasound system according to various embodiments.

FIG. 1C is a schematic illustration of an ultrasound system according to various embodiments.

FIG. 2 is a schematic illustration of an ultrasound system coupled to a region of interest according to various embodiments.

FIG. 3 is a schematic representation of an imaging diagnostic ultrasound system according to various embodiments.

FIG. 4 is a schematic representation of bidirectional imaging at the same lateral location according to various embodiments.

FIG. 5 is a schematic representation of directionally dependent focal zone sequencing according to various embodiments.

FIG. 6 is a schematic representation of directionally dependent focal zone sequencing with different triggering locations according to various embodiments.

FIG. 7 is a schematic representation of directionally dependent focal zone sequencing on consecutive A-lines according to various embodiments.

FIGS. 8A and 8B are a graph and schematic image of production of a multipath echo artifact over time according to various embodiments.

FIGS. 9A and 9B are a graph and schematic image of reducing or eliminating a multipath echo artifact using static wait times according to various embodiments.

FIGS. 10A and 10B are a graph and schematic image of production of a multipath echo artifact with a dynamic, or changing offset gap according to various embodiments.

FIG. 11 illustrates a method of reducing or eliminating artifact in a dynamic offset that changes over time according to various embodiments.

FIG. 12A is a schematic representation of multiple focal zone imaging that produces an artifact in one or more focal zones according to one embodiment.

FIG. 12B is a schematic representation of multiple blended focal zone imaging reduces or eliminates a display of an artifact in one or more focal zones according to one embodiment.

FIG. 13 illustrates a method to determine ingress image trigger offsets to improve lateral imaging alignment registration according to various embodiments.

FIG. 14 is a captured image of unstable pixels shaking according to various embodiments.

FIG. 15A is a quantified temporal motion artifact with a predominantly lateral shift according to various embodiments.

FIG. 15B is a quantified temporal motion artifact temporally stable according to various embodiments.

FIG. 15C is a quantified temporal motion artifact that is uniform in depth according to various embodiments.

FIG. 16A is a captured image of unstable pixels shaking in a lateral direction according to various embodiments.

FIG. 16B is a captured image using a shift filter to stabilize the image according to various embodiments.

FIG. 17A is a captured image of unstable pixels shaking in an elevational direction according to various embodiments.

FIG. 17B is a captured image using temporally average consecutive frames filter according to various embodiments.

FIG. 18A is a captured image of unstable pixels shaking according to various embodiments.

FIG. 18B is a captured image using shift data and temporally average consecutive frames filter according to various embodiments.

FIG. 19 is diagram illustrating the calculated correlation coefficient over time according to various embodiments.

FIG. 20 is a diagram illustrating frame to frame motion detection according to various embodiments.

FIG. 21 is a diagram illustrating the calculated correlation coefficient over time according to various embodiments.

FIG. 22A is a captured image of unstable pixels shaking according to various embodiments.

FIG. 22B is a captured image using shift data and temporally average consecutive frames filter when no motion is detected according to various embodiments.

### DETAILED DESCRIPTION

The following description sets forth examples of embodiments, and is not intended to limit the present invention or its teachings, applications, or uses thereof. It should be understood that throughout the drawings, corresponding reference numerals indicate like or corresponding parts and features. The description of specific examples indicated in various embodiments are intended for purposes of illustration only and are not intended to limit the scope of the invention disclosed herein. Moreover, recitation of multiple embodiments having stated features is not intended to exclude other embodiments having additional features or other embodiments incorporating different combinations of the stated features. Further, features in one embodiment (such as in one figure) may be combined with descriptions (and figures) of other embodiments.

In various embodiments, systems and methods for ultrasound imaging of tissue are adapted for and/or configured to use one or more focal zones in the tissue for imaging. In one embodiment, one focal zone is used for imaging. In various embodiments, two, three, four, or more focal zones are used for imaging. In various embodiments, an ultrasound transducer for imaging is placed directly in contact through acoustic coupling to a tissue such as a skin surface for imaging the one or more focal zones under the skin surface. In various embodiments, an ultrasound transducer for imaging has an offset gap between the imaging transducer and a portion of a housing (such as at an acoustic transmission window, such as a PEEK window) in an ultrasound probe, whereby the portion of the housing is placed in contact through acoustic coupling to a tissue such as a skin surface for imaging the one or more focal zones under the skin surface. In some embodiments, an ultrasound transducer for imaging has an offset gap between the imaging transducer and a portion of a housing that uses two or more (e.g., 2, 3, 4, 5, 6, or more) focal zones can produce multipath artifacts from acoustic ultrasound energy that bounces between the imaging transducer and (i) the acoustic window and/or (ii) the region being imaged. These artifacts may obscure the clarity of the imaging. In various embodiments described herein, systems and methods reduce and/or eliminate such artifacts. In some embodiments, imaging is stationary (e.g., the tissue and/or at least a part of the device is not moving) In some embodiments, imaging is in motion (e.g., the tissue and/or at least a part of the device is moving).

In various embodiments, ultrasound imaging is used to visualize a tissue region and/or anatomy. In one embodiment, ultrasound imaging is used to confirm sufficient acoustic coupling to a tissue region for improving imaging correlation between movement of the ultrasound imaging transducer in a first and second direction when forming images.

In various embodiments, ultrasound imaging is used in conjunction with a cosmetic treatment or a medical treatment in order to visualize, plan and/or monitor the cosmetic or medical treatment. In one embodiment, ultrasound imaging is used in conjunction with an application of energy to a tissue. In one embodiment, ultrasound imaging is used in conjunction with an application of ultrasound therapy to a tissue. In one embodiment, ultrasound imaging is used in conjunction with an application of a dermal filler to a tissue. In one embodiment, ultrasound imaging is used in conjunction with an application of a drug or a compound to a tissue. In one embodiment, ultrasound imaging is used in conjunction with an application of a botulinum toxin to a tissue.

In various embodiments, systems and methods for ultrasound treatment of tissue are adapted for and/or configured to provide cosmetic treatment. In some embodiments, devices and methods of directing ultrasound therapy to a single focus point or multiple, simultaneous focus points. In various embodiments, ultrasound imaging is used to confirm sufficient acoustic coupling to a treatment area for improving performance or providing improved correlation between movement in a first and second direction when forming images in cosmetic and/or medical procedures. In some embodiments, devices and methods of employing ultrasound imaging to confirm sufficient acoustic coupling to a treatment area for improving performance and safety when directing ultrasound therapy to a single focus point or multiple, simultaneous focus points in cosmetic and/or medical procedures. In some embodiments, devices and methods of improved ultrasound imaging provide better correlation between movement in a first and second direction when forming images. Embodiments of the invention provide better imaging correlation between a first moving direction and a second moving direction, (e.g., better correlation between left-traveling & right-traveling formed images). Embodiments of the invention provide better spatial registration between a first moving direction and a second moving direction, (e.g., better correlation between left-traveling & right-traveling formed images). Devices and methods of improved ultrasound imaging improve effect A-lines and/or B-mode imaging faster (e.g., 1.5×, 2×, 3×, 5× times the scanning rate). In various embodiments, tissue below or even at a skin surface such as epidermis, dermis, fascia, muscle, fat, and superficial muscular aponeurotic system (“SMAS”), are treated non-invasively with ultrasound energy. The ultrasound energy can be focused at one or more treatment points and/or zones, can be unfocused and/or defocused, and can be applied to a region of interest containing at least one of epidermis, dermis, hypodermis, fascia, muscle, fat, cellulite, and SMAS to achieve a cosmetic and/or therapeutic effect. In various embodiments, systems and/or methods provide non-invasive dermatological treatment to tissue through thermal treatment, coagulation, ablation, and/or tightening. In several embodiments disclosed herein, non-invasive ultrasound is used to achieve one or more of the following effects: a face lift, a brow lift, a chin lift, an eye treatment (e.g., malar bags, treat infraorbital laxity), a wrinkle reduction, fat reduction (e.g., treatment of adipose and/or cellulite), cellulite treatment (e.g., dimple or non-dimple type female gynoid lipodystrophy), décolletage improvement (e.g, upper chest), a buttock lift (e.g, buttock tightening), a skin laxity treatment (e.g, treatment of tissue for tightening or an abdominal laxity treatment), a scar reduction, a burn treatment, a tattoo removal, a vein removal, a vein reduction, a treatment on a sweat gland, a treatment of hyperhidrosis, sun spot removal, an acne treatment, and a pimple removal. In one embodiment, fat reduction is achieved. In various embodiments, cellulite (e.g., dimple or non-dimple type gynoid lipodystrophy) reduction or amelioration of one or more characteristics (such as dimples, nodularity, “orange peel” appearance, etc., is achieved by about 10-20%, 20-40%, 40-60%, 60-80% or higher (as well as overlapping ranging therein) as compared to, for example, untreated tissue. In one embodiment, décolletage is treated. In some embodiments, two, three or more beneficial effects are achieved during the same treatment session, and may be achieved simultaneously.

Various embodiments relate to devices or methods of controlling the delivery of energy to tissue. In various embodiments, various forms of energy can include acoustic, ultrasound, light, laser, radio-frequency (RF), microwave, electromagnetic, radiation, thermal, cryogenic, electron beam, photon-based, magnetic, magnetic resonance, and/or other energy forms. Various embodiments relate to devices or methods of splitting an ultrasonic energy beam into multiple beams. In various embodiments, devices or methods can be used to alter the delivery of ultrasound acoustic energy in any procedures such as, but not limited to, therapeutic ultrasound, diagnostic ultrasound, ultrasonic welding, any application that involves coupling mechanical waves to an object, and other procedures. Generally, with therapeutic ultrasound, a tissue effect is achieved by concentrating the acoustic energy using focusing techniques from the aperture. In some instances, high intensity focused ultrasound (HIFU) is used for therapeutic purposes in this manner. In one embodiment, a tissue effect created by application of therapeutic ultrasound at a particular depth can be referred to as creation of a thermal coagulation point (TCP). In some embodiments, a zone can include a point. In some embodiments, a zone is a line, plane, spherical, elliptical, cubical, or other one-, two-, or three-dimensional shape. It is through creation of TCPs at particular positions that thermal and/or mechanical ablation of tissue can occur non-invasively or remotely. In some embodiments, an ultrasound treatment does not include cavitation and/or shock waves. In some embodiments, an ultrasound treatment includes cavitation and/or shock waves.

In one embodiment, TCPs can be created in a linear or substantially linear, curved or substantially curved, zone or sequence, with each individual TCP separated from neighboring TCPs by a treatment spacing. In one embodiment, multiple sequences of TCPs can be created in a treatment region. For example, TCPs can be formed along a first sequence and a second sequence separated by a treatment distance from the first sequence. Although treatment with therapeutic ultrasound can be administered through creation of individual TCPs in a sequence and sequences of individual TCPs, it may be desirable to reduce treatment time and corresponding risk of pain and/or discomfort experienced by a patient. Therapy time can be reduced by forming multiple TCPs simultaneously, nearly simultaneously, or sequentially. In some embodiments, a treatment time can be reduced 10%, 20%, 25%, 30%, 35%, 40%, 45%, 50%, 55%, 60%, 65%, 70%, 75%, 80% or more by creating multiple TCPs.

Various embodiments address potential challenges posed by administration of ultrasound therapy. In various embodiments, time for effecting the formation of TCPs for a desired cosmetic and/or therapeutic treatment for a desired clinical approach at a target tissue is reduced. In various embodiments, target tissue is, but is not limited to, any of skin, eyelids, eye lash, eye brow, caruncula lacrimalis, crow's feet, wrinkles, eye, nose, mouth (e.g., nasolabial fold, perioral wrinkles), tongue, teeth, gums, ears, brain, heart, lungs, ribs, abdomen (e.g, for abdominal laxity), stomach, liver, kidneys, uterus, breast, vagina, prostrate, testicles, glands, thyroid glands, internal organs, hair, muscle, bone, ligaments, cartilage, fat, fat labuli, adipose tissue, subcutaneous tissue, implanted tissue, an implanted organ, lymphoid, a tumor, a cyst, an abscess, or a portion of a nerve, or any combination thereof.

Various embodiments of ultrasound treatment and/or imaging devices are described in U.S. application Ser. No. 12/996,616, which published as U.S. Publication No. 2011-0112405 A1 on May 12, 2011, which is a U.S. National Phase under 35 U.S.C. § 371 of International Application No. PCT/US2009/046475, filed on Jun. 5, 2009 and published in English on Dec. 10, 2009, each of which is incorporated in its entirety by reference, herein. Various embodiments of ultrasound treatment and/or imaging devices are described in U.S. application Ser. No. 14/193,234, which published as U.S. Publication No. 2014/0257145 on Sep. 11, 2014, which is incorporated in its entirety by reference, herein. Various embodiments of ultrasound treatment and/or imaging devices are described in International App. PCT/US17/46703, which published as WO 2018/035012 on Feb. 22, 2018 with a national phase U.S. application Ser. No. 15/562,384, which published as U.S. Publication No. 2019/0142380 on May 16, 2019, each of which is incorporated in its entirety by reference, herein. Various embodiments of ultrasound treatment and/or imaging devices are described in International App. PCT/US19/14617, which published as WO 2019/147596 on Aug. 1, 2019 with a national phase U.S. application Ser. No. 16/964,914, which published as U.S. Publication No. 2021/0038925 on Feb. 11, 2021, each of which is incorporated in its entirety by reference, herein.

### System Overview

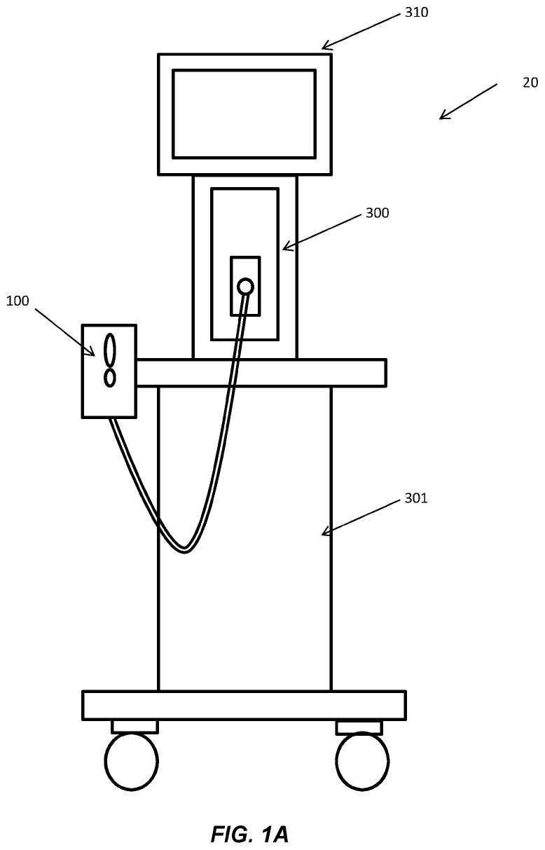

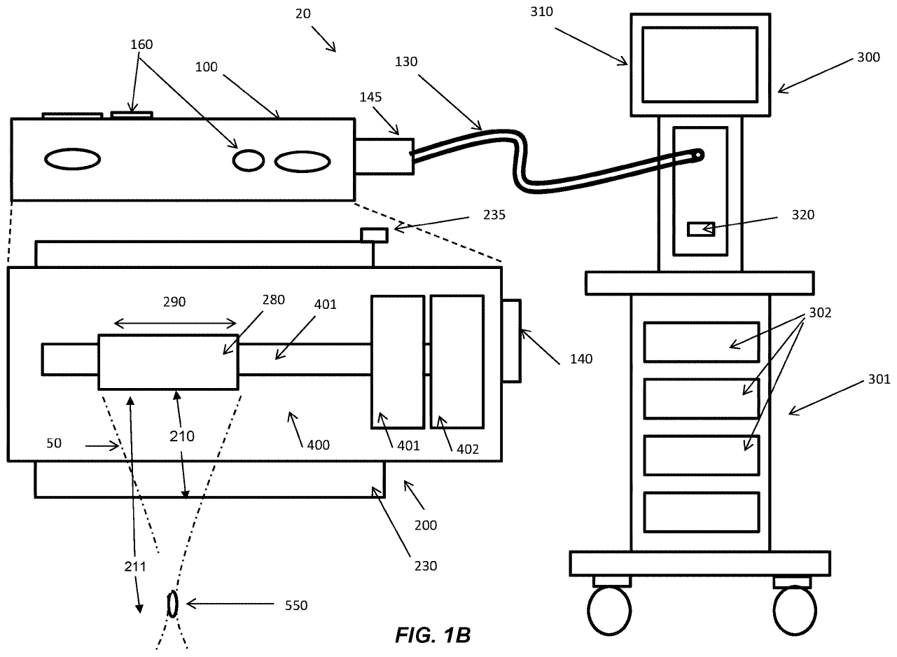

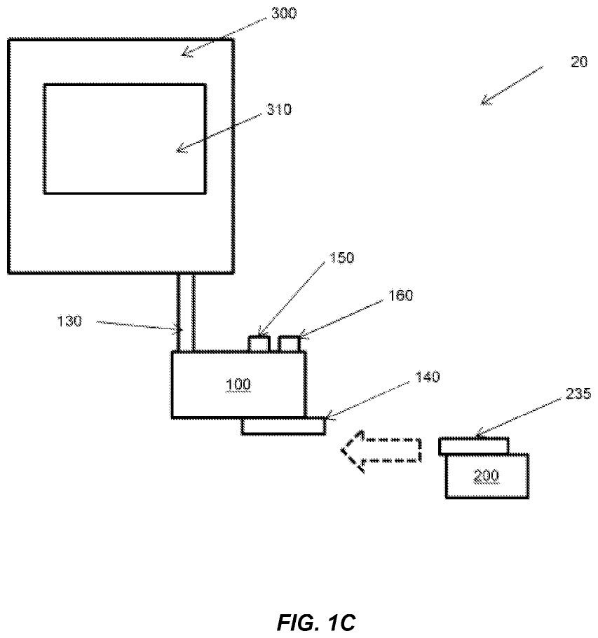

With reference to the illustration in FIGS. 1A, 1B, and 1C, various embodiments of an ultrasound system 20 includes a hand wand (e.g., handpiece) 100, module (e.g., transducer module, cartridge, probe) 200, and a controller (e.g., console) 300. In some embodiments, a console 300 comprises a communication system (e.g., wifi, Bluetooth, modem, etc. to communicate with another party, a manufacturer, a supplier, a service provider, the Internet, and/or a cloud. In some embodiments, a cart 301 provides mobility and/or position of the system 20, and can include wheels, surfaces to write on or place components, and/or compartments 302 (e.g., drawers, containers, shelves, etc.) to, for example, store or organize components. In some embodiments, the cart has a power supply, such as a power connection to a battery and/or one or more cords to connect power, communications (e.g., Ethernet) to the system 20. In some embodiments, the system 20 comprises a cart 301. In some embodiments, the system 20 does not comprise a cart 301. The hand wand 100 can be coupled to the controller 300 by an interface 130, which may be a wired or wireless interface. The interface 130 can be coupled to the hand wand 100 by a connector 145. The distal end of the interface 130 can be connected to a controller connector on a circuit 345 (not shown). In one embodiment, the interface 130 can transmit controllable power from the controller 300 to the hand wand 100. In an embodiment, the system 20 has multiple imaging channels (e.g., 2, 4, 6, 8, 10 channels) for ultra-clear HD (high definition) visualization of subcutaneous structures to improve imaging. In an embodiment, the system 20 has multiple therapy channels (e.g., 2, 4, 6, 8, 10 channels) and a precision linear-drive motor that doubles treatment accuracy while increasing speed (e.g., by 25%, 40%, 50%, 60%, 75%, 100% or more).

In various embodiments, the controller 300 can be adapted to and/or configured for operation with the hand wand 100 and the module 200, as well as the overall ultrasound system 20 functionality. In various embodiments, multiple controllers 300, 300′, 300″, etc. can be adapted to and/or configured for operation with multiple hand wands 100, 100′, 100″, etc. and or multiple modules 200, 200′, 200″, etc. The controller 300 can include connectivity to one or more interactive graphical display 310, which can include a touchscreen monitor and Graphic User Interface (GUI) that allows the user to interact with the ultrasound system 20. In one embodiment, a second smaller, more mobile display allows the user to more easily position and view the treatment screen. In one embodiment, a second display allows the system user to view a treatment screen (e.g., on a wall, mobile device, large screen, remote screen). In one embodiment the graphical display 310 includes a touchscreen interface 315 (not shown). In various embodiments, the display 310 sets and displays the operating conditions, including equipment activation status, treatment parameters, system messages and prompts, and ultrasound images. In various embodiments, the controller 300 can be adapted to and/or configured to include, for example, a microprocessor with software and input/output devices, systems and devices for controlling electronic and/or mechanical scanning and/or multiplexing of transducers and/or multiplexing of transducer modules, a system for power delivery, systems for monitoring, systems for sensing the spatial position of the probe and/or transducers and/or multiplexing of transducer modules, and/or systems for handling user input and recording treatment results, among others. In various embodiments, the controller 300 can include a system processor and various analog and/or digital control logic, such as one or more of microcontrollers, microprocessors, field-programmable gate arrays, computer boards, and associated components, including firmware and control software, which may be capable of interfacing with user controls and interfacing circuits as well as input/output circuits and systems for communications, displays, interfacing, storage, documentation, and other useful functions. System software running on the system process may be adapted to and/or configured to control all initialization, timing, level setting, monitoring, safety monitoring, and all other ultrasound system functions for accomplishing user-defined treatment objectives. Further, the controller 300 can include various input/output modules, such as switches, buttons, etc., that may also be suitably adapted to and/or configured to control operation of the ultrasound system 20.

In one embodiment, the hand wand 100 includes one or more finger activated controllers or switches, such as 150 and 160. In various embodiments, one or more thermal treatment controllers 160 (e.g., switch, button) activates and/or stops treatment. In various embodiments, one or more imaging controllers 150 (e.g., switch, button) activates and/or stops imaging. In one embodiment, the hand wand 100 can include a removable module 200. In other embodiments, the module 200 may be non-removable. In various embodiments, the module 200 can be mechanically coupled to the hand wand 100 using a latch or coupler 140. In various embodiments, an interface guide 235 or multiple interface guides 235 can be used for assisting the coupling of the module 200 to the hand wand 100. The module 200 can include one or more ultrasound transducers 280. In some embodiments, an ultrasound transducer 280 includes one or more ultrasound elements. The module 200 can include one or more ultrasound elements. In one embodiment, the module 200 comprises a bubble trap to reduce bubbles in an acoustic medium. The hand wand 100 can include imaging-only modules, treatment-only modules, imaging-and-treatment modules, and the like. In various embodiments, the ultrasound transducer 280 is movable in one or more directions 290 within the module 200. In some embodiments, the transducer 280 is connected to a motion mechanism 400. In some embodiments, the transducer 280 is not connected to a motion mechanism 400. In various embodiments, the motion mechanism comprises zero, one, or more bearings, shafts, rods, screws, lead screws 401, encoders 402 (e.g., optical encoder to measure position of the transducer 280), motors 403 (e.g., a step motor) to help ensure accurate and repeatable movement of the transducer 280 within the module 200. In various embodiments, module 200 can include a transducer 280 which can emit energy through an acoustically transparent member 230. In one embodiment, the module 200 has an offset distance 210 between the transducer 280 and the acoustically transparent member 230. In one embodiment, the module 200 has an offset distance 211 between the transducer 280 and bottom of an imaging region distance. In one embodiment, the control module 300 can be coupled to the hand wand 100 via the interface 130, and the graphic user interface 310 can be adapted to and/or configured for controlling the module 200. In one embodiment, the control module 300 can provide power to the hand wand 100. In one embodiment, the hand wand 100 can include a power source. In one embodiment, the switch 150 can be adapted to and/or configured for controlling a tissue imaging function and the switch 160 can be adapted to and/or configured for controlling a tissue treatment function. In various embodiments, delivery of emitted energy 50 at a suitable focal depth, distribution, timing, and energy level is provided by the module 200 through controlled operation by the control system 300 of the transducer 280 to achieve the desired therapeutic effect with a thermal coagulation zone 550.

In one embodiment, the module 200 can be coupled to the hand wand 100. The module 200 can emit and receive energy, such as ultrasonic energy. The module 200 can be electronically coupled to the hand wand 100 and such coupling may include an interface which is in communication with the controller 300. In one embodiment, the interface guide 235 can be adapted to and/or configured to provide electronic communication between the module 200 and the hand wand 100. The module 200 can comprise various probe and/or transducer configurations. For example, the module 200 can be adapted to and/or configured for a combined dual-mode imaging/therapy transducer, coupled or co-housed imaging/therapy transducers, separate therapy and imaging probes, and the like. In one embodiment, when the module 200 is inserted into or connected to the hand wand 100, the controller 300 automatically detects it and updates the interactive graphical display 310.

In some embodiments, an access key 320 (e.g., a secure USB drive, key) is removably connected to a system 20 to permit the system 20 to function. In various embodiments, the access key is programmed to be customer specific, and serves multiple functions, including system security, country/region specific access to treatment guidelines and functionality, software upgrades, support log transfers and/or credit transfer and/or storage. In various embodiments, the system 20 has internet and/or data connectivity. In an embodiment, connectivity provides a method by which data is transferred between the system 20 provider and the customer. In various embodiments, data includes credits, software updates and support logs. Connectivity is divided into different model embodiments, based on how a user's console is connected to the internet. In one embodiment, Disconnected Model connectivity comprises a console that is disconnected from the internet and customer doesn't have internet access. Credit transfers and software upgrades are conducted by shipping access key(s), (e.g., USB drives) to the customer. In one embodiment, Semi-Connected Model connectivity comprises a console that is disconnected from the internet but customer has internet access. Credit transfers, software upgrades and support log transfers are conducted using the customer's personal computer, smart phone, or other computing device in conjunction with the system access key to transfer data. In one embodiment, Fully-Connected Model connectivity comprises a console that is wirelessly connected to the internet using wifi, cellular modem, Bluetooth, or other protocol. Credit transfers, software upgrades and support log transfers are made directly between the console and the cloud. In various embodiments, the system 20 connects to an online portal, for streamlined and/or automated inventory management, on-demand treatment purchases and business analytics insights to drive customer aesthetic treatment business to the next level.

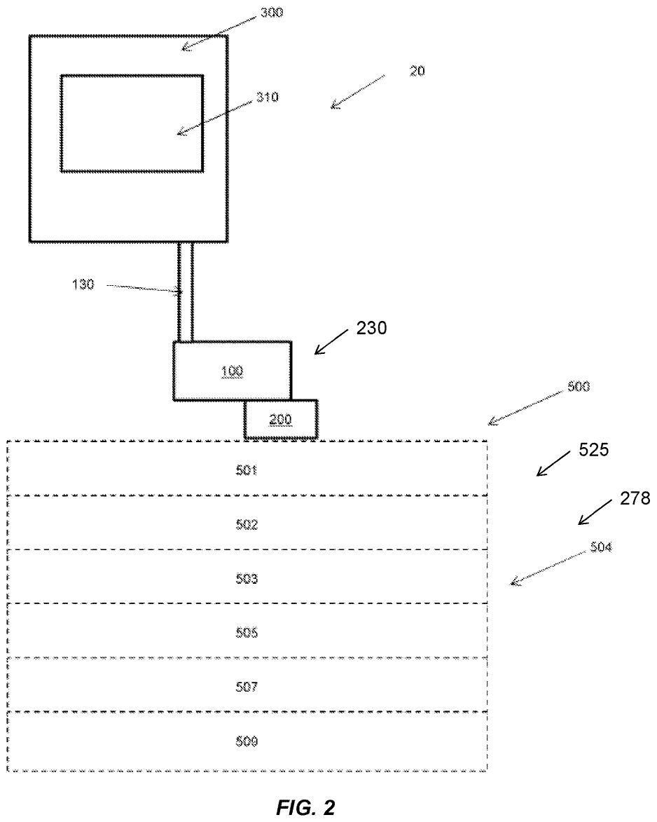

In various embodiments, tissue below or even at a skin surface such as epidermis, dermis, hypodermis, fascia, and superficial muscular aponeurotic system (“SMAS”), and/or muscle are treated non-invasively with ultrasound energy. Tissue may also include blood vessels and/or nerves. The ultrasound energy can be focused, unfocused or defocused and applied to a region of interest containing at least one of epidermis, dermis, hypodermis, fascia, and SMAS to achieve a therapeutic effect. FIG. 2 is a schematic illustration of the ultrasound system 20 coupled to a region of interest 10. In various embodiments, tissue layers of the region of interest 10 can be at any part of the body of a subject. In one embodiment, the tissue layers are in the head and face region of the subject. The cross-sectional portion of the tissue of the region of interest 10 includes a skin surface 501, an epidermal layer 502, a dermal layer 503, a fat layer 505, a superficial muscular aponeurotic system 507 (hereinafter “SMAS 507”), and a muscle layer 509. The tissue can also include the hypodermis 504, which can include any tissue below the dermal layer 503. The combination of these layers in total may be known as subcutaneous tissue 510. Also illustrated in FIG. 2 is a treatment zone 525 which is below the surface 501. In one embodiment, the surface 501 can be a surface of the skin of a subject 500. Although an embodiment directed to therapy at a tissue layer may be used herein as an example, the system can be applied to any tissue in the body. In various embodiments, the system and/or methods may be used on tissue (including but not limited to one or a combination of muscles, fascia, SMAS, dermis, epidermis, fat, adipose cells, cellulite, which may be called gynoid lipodystrophy, (e.g., non-dimple type female gynoid lipodystrophy), collagen, skin, blood vessels, of the face, neck, head, arms, legs, or any other location on or in the body (including bodily cavities). In various embodiments, cellulite (e.g., non-dimple type female gynoid lipodystrophy) reduction is achieved in an amount of 2%, 5%, 10%, 15%, 20%, 25%, 30%, 40%, 50%, 75%, 80%, 90%, 95%, and any ranges therein.

With reference to the illustration in FIG. 2, an embodiment of the ultrasound system 20 includes the hand wand 100, the module 200, and the controller 300. In one embodiment, the module 200 includes a transducer 280. In one embodiment, an ultrasound system 20 with a transducer 280 adapted to and/or configured to treat tissue at a focal depth 278. In one embodiment, the focal depth 278 is a distance between the transducer 280 and the target tissue for treatment. In one embodiment, a focal depth 278 is fixed for a given transducer 280. In one embodiment, a focal depth 278 is variable for a given transducer 280. In one embodiment, a transducer 280 is configured to treat simultaneously at multiple depths below a skin surface (e.g., 1.5 mm, 3.0 mm, 4.5 mm, or other depths).

In one embodiment, the module 200 can include a transducer 280 which can emit energy through an acoustically transparent member 230. In various embodiments, a depth may refer to the focal depth 278. In one embodiment, the transducer 280 can have an offset distance 270, which is the distance between the transducer 280 and a surface of the acoustically transparent member 230. In one embodiment, the focal depth 278 of a transducer 280 is a fixed distance from the transducer. In one embodiment, a transducer 280 may have a fixed offset distance 270 from the transducer to the acoustically transparent member 230. In one embodiment, an acoustically transparent member 230 is adapted to and/or configured at a position on the module 200 or the ultrasound system 20 for contacting the skin surface 501. In various embodiments, the focal depth 278 exceeds the offset distance 270 by an amount to correspond to treatment at a target area located at a tissue depth 279 below a skin surface 501. In various embodiments, when the ultrasound system 20 placed in physical contact with the skin surface 501, the tissue depth 279 is a distance between the acoustically transparent member 230 and the target area, measured as the distance from the portion of the hand wand 100 or module 200 surface that contacts skin (with or without an acoustic coupling gel, medium, etc.) and the depth in tissue from that skin surface contact point to the target area. In one embodiment, the focal depth 278 can correspond to the sum of an offset distance 270 (as measured to the surface of the acoustically transparent member 230 in contact with a coupling medium and/or skin 501) in addition to a tissue depth 279 under the skin surface 501 to the target region. In various embodiments, the acoustically transparent member 230 is an acoustic window, such as a PEEK window configured for transmitting ultrasound through a coupling medium (or media) within the module 200 to the outside of the acoustically transparent member 230.

Coupling components can comprise various substances, materials, and/or devices to facilitate coupling of the transducer 280 or module 200 to a region of interest. For example, coupling components can comprise an acoustic coupling system adapted to and/or configured for acoustic coupling of ultrasound energy and signals. Acoustic coupling system with possible connections such as manifolds may be utilized to couple sound into the region of interest, provide liquid- or fluid-filled lens focusing. The coupling system may facilitate such coupling through use of one or more coupling media, including air, gases, water, liquids, fluids, gels, solids, non-gels, and/or any combination thereof, or any other medium that allows for signals to be transmitted between the transducer 280 and a region of interest. In one embodiment one or more coupling media is provided inside a transducer. In one embodiment a fluid-filled module 200 contains one or more coupling media inside a housing. In one embodiment a fluid-filled module 200 contains one or more coupling media inside a sealed housing, which is separable from a dry portion of an ultrasonic device. In various embodiments, a coupling medium is used to transmit ultrasound energy between one or more devices and tissue with a transmission efficiency of 100%, 99% or more, 98% or more, 95% or more, 90% or more, 80% or more, 75% or more, 60% or more, 50% or more, 40% or more, 30% or more, 25% or more, 20% or more, 10% or more, and/or 5% or more.

In various embodiments, the transducer 280 can image and treat a region of interest at any suitable tissue depths 279. In one embodiment, the transducer module 280 can provide an acoustic power in a range of about 1 W or less, between about 1 W to about 100 W, and more than about 100 W, e.g., 200 W, 300 W, 400 W, 500 W. In one embodiment, the transducer module 280 can provide an acoustic power at a frequency of about 1 MHz or less, between about 1 MHz to about 10 MHz (e.g., 3 MHz, 4 MHz, 4.5 MHz, 7 MHz, 10 MHz), and more than about 10 MHz. In one embodiment, the module 200 has a focal depth 278 for a treatment at a tissue depth 279 of about 4.5 mm below the skin surface 501. In one embodiment, the module 200 has a focal depth 278 for a treatment at a tissue depth 279 of about 3 mm below the skin surface 501. In one embodiment, the module 200 has a focal depth 278 for a treatment at a tissue depth 279 of about 1.5 mm below the skin surface 501. Some non-limiting embodiments of transducers 280 or modules 200 can be adapted to and/or configured for delivering ultrasonic energy at a tissue depth of 1.5 mm, 3 mm, 4.5 mm, 6 mm, 7 mm, less than 3 mm, between 3 mm and 4.5 mm, between 4.5 mm and 6 mm, more than more than 4.5 mm, more than 6 mm, etc., and anywhere in the ranges of 0-3 mm, 0-4.5 mm, 0-6 mm, 0-25 mm, 0-100 mm, etc. and any depths therein. In one embodiment, the ultrasound system 20 is provided with two or more transducer modules 280. For example, a first transducer module can apply treatment at a first tissue depth (e.g., 4.5 mm) and a second transducer module can apply treatment at a second tissue depth (e.g., 3 mm), and a third transducer module can apply treatment at a third tissue depth (e.g., 1.5-2 mm). In one embodiment, at least some or all transducer modules can be adapted to and/or configured to apply treatment at substantially same depths.

In various embodiments, changing the number of focus point locations (e.g., such as with a tissue depth 279) for an ultrasonic procedure can be advantageous because it permits treatment of a patient at varied tissue depths even if the focal depth 278 of a transducer 270 is fixed. This can provide synergistic results and maximizing the clinical results of a single treatment session. For example, treatment at multiple depths under a single surface region permits a larger overall volume of tissue treatment, which results in enhanced collagen formation and tightening. Additionally, treatment at different depths affects different types of tissue, thereby producing different clinical effects that together provide an enhanced overall cosmetic result. For example, superficial treatment may reduce the visibility of wrinkles and deeper treatment may induce formation of more collagen growth. Likewise, treatment at various locations at the same or different depths can improve a treatment.

Although treatment of a subject at different locations in one session may be advantageous in some embodiments, sequential treatment over time may be beneficial in other embodiments. For example, a subject may be treated under the same surface region at one depth in time one, a second depth in time two, etc. In various embodiments, the time can be on the order of nanoseconds, microseconds, milliseconds, seconds, minutes, hours, days, weeks, months, or other time periods. The new collagen produced by the first treatment may be more sensitive to subsequent treatments, which may be desired for some indications. Alternatively, multiple depth treatment under the same surface region in a single session may be advantageous because treatment at one depth may synergistically enhance or supplement treatment at another depth (due to, for example, enhanced blood flow, stimulation of growth factors, hormonal stimulation, etc.). In several embodiments, different transducer modules provide treatment at different depths. In one embodiment, a single transducer module can be adjusted or controlled for varied depths. Safety features to minimize the risk that an incorrect depth will be selected can be used in conjunction with the single module system.

In several embodiments, a method of treating the lower face and neck area (e.g., the submental area) is provided. In several embodiments, a method of treating (e.g., softening) mentolabial folds is provided. In other embodiments, a method of treating the eye region (e.g., malar bags, treat infraorbital laxity) is provided. Upper lid laxity improvement and periorbital lines and texture improvement will be achieved by several embodiments by treating at variable depths. By treating at varied locations in a single treatment session, optimal clinical effects (e.g., softening, tightening) can be achieved. In several embodiments, the treatment methods described herein are non-invasive cosmetic procedures. In some embodiments, the methods can be used in conjunction with invasive procedures, such as surgical facelifts or liposuction, where skin tightening is desired. In various embodiments, the methods can be applied to any part of the body.

In one embodiment, a transducer module 200 permits a treatment sequence at a fixed depth at or below the skin surface. In one embodiment, a transducer module permits a treatment sequence at one, two, or more variable or fixed depths below the dermal layer. In several embodiments, the transducer module comprises a movement mechanism adapted to and/or configured to direct ultrasonic treatment in a sequence of individual thermal lesions (hereinafter “thermal coagulation points” or “TCPs”) at a fixed focal depth. In one embodiment, the sequence of individual TCPs has a treatment spacing in a range from about 0.01 mm to about 25 mm (e.g., 1 mm, 1.5 mm, 2 mm, 2.5 mm, 3 mm, 5 mm, 10 mm, 20 mm and any value ranges therein), with a dithering alteration of the spacing by 1-50% (e.g., 1%, 5%, 10%, 15%, 20%, 25%, 30%, 35%, 40%, 45%, 50% and any range therein). For example, the spacing can be 1.1 mm or less, 1.5 mm or more, between about 1.1 mm and about 1.5 mm, etc. In one embodiment, the individual TCPs are discrete. In one embodiment, the individual TCPs are overlapping. In one embodiment, the movement mechanism is adapted to and/or configured to be programmed to provide variable spacing between the individual TCPs. In one embodiment, the dithering can be adapted to and/or configured to provide variable spacing between the individual TCPs. In several embodiments, a transducer module comprises a movement mechanism adapted to and/or configured to direct ultrasonic treatment in a sequence so that TCPs are formed in linear or substantially linear sequences separated by a treatment distance. For example, a transducer module can be adapted to and/or configured to form TCPs along a first linear sequence and a second linear sequence separated by a treatment distance from the first linear sequence. In one embodiment, treatment distance between adjacent linear sequences of individual TCPs is in a range from about 0.01 mm to about 25 mm. In one embodiment, treatment distance between adjacent linear sequences of individual TCPs is in a range from about 0.01 mm to about 50 mm. For example, the treatment distance can be 2 mm or less, 3 mm or more, between about 2 mm and about 3 mm, etc. In several embodiments, a transducer module can comprise one or more movement mechanisms 400 adapted to and/or configured to direct ultrasonic treatment in a sequence so that TCPs are formed in linear or substantially linear sequences of individual thermal lesions separated by a treatment distance from other linear sequences. In one embodiment a treatment is applied in a first direction 290 (e.g., push). In one embodiment, a treatment is applied opposite the first direction 290 (e.g., pull). In one embodiment, treatment is applied in both a first direction 290 and opposite the first direction (e.g., push and pull). In one embodiment, the treatment distance separating linear or substantially linear TCPs sequences is the same or substantially the same. In one embodiment, the treatment distance separating linear or substantially linear TCPs sequences is different or substantially different for various adjacent pairs of linear TCPs sequences.

In one embodiment, first and second removable transducer modules are provided. In one embodiment, each of the first and second transducer modules are adapted to and/or configured for both ultrasonic imaging and ultrasonic treatment. In one embodiment, a transducer module is adapted to and/or configured for treatment only. In one embodiment, an imaging transducer may be attached to a handle of a probe or a hand wand. The first and second transducer modules are adapted to and/or configured for interchangeable coupling to a hand wand. The first transducer module is adapted to and/or configured to apply ultrasonic therapy to a first layer of tissue, while the second transducer module is adapted to and/or configured to apply ultrasonic therapy to a second layer of tissue. The second layer of tissue is at a different depth than the first layer of tissue.

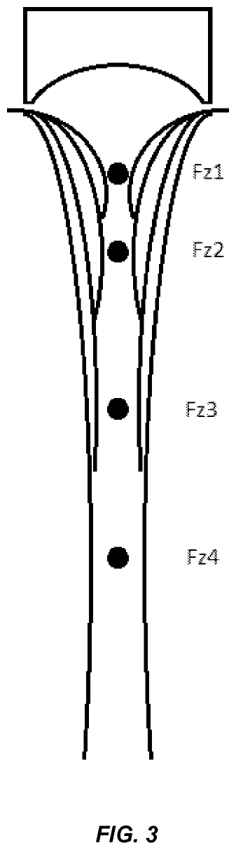

In various embodiments, delivery of emitted energy 50 at a suitable focal depth 278, distribution, timing, and energy level is provided by the module 200 through controlled operation by the control system 300 to achieve the desired therapeutic effect of controlled thermal injury to treat at least one of the epidermis layer 502, dermis layer 503, fat layer 505, the SMAS layer 507, the muscle layer 509, and/or the hypodermis 504. FIG. 3 illustrates one embodiment of a depth that corresponds to a depth for treating muscle. In various embodiments, the depth can correspond to any tissue, tissue layer, skin, epidermis, dermis, hypodermis, fat, SMAS, muscle, blood vessel, nerve, or other tissue. During operation, the module 200 and/or the transducer 280 can also be mechanically and/or electronically scanned along the surface 501 to treat an extended area. Before, during, and after the delivery of ultrasound energy 50 to at least one of the epidermis layer 502, dermis layer 503, hypodermis 504, fat layer 505, the SMAS layer 507 and/or the muscle layer 509, monitoring of the treatment area and surrounding structures can be provided to plan and assess the results and/or provide feedback to the controller 300 and the user via a graphical interface 310.

In one embodiment, an ultrasound system 20 generates ultrasound energy which is directed to and focused below the surface 501. This controlled and focused ultrasound energy 50 creates the thermal coagulation point or zone (TCP) 550. In one embodiment, the ultrasound energy 50 creates a void in subcutaneous tissue 510. In various embodiments, the emitted energy 50 targets the tissue below the surface 501 which cuts, ablates, coagulates, micro-ablates, manipulates, and/or causes a TCP 550 in the tissue portion 10 below the surface 501 at a specified focal depth 278. In one embodiment, during the treatment sequence, the transducer 280 moves in a direction denoted by the arrow marked 290 at specified intervals 295 to create a series of treatment zones 254 each of which receives an emitted energy 50 to create one or more TCPs 550. In one embodiment, an arrow marked 291 illustrates an axis or direction that is orthogonal to arrow 290, and a spacing of TCP's 550 show TCP's can be spaced orthogonally to the motion direction of the transducer 280. In some embodiments, an orientation of the spaced TCP's can be set at any angle 0-180 degrees from arrow 290. In some embodiments, an orientation of the spaced TCP's can be set at any angle 0-180 degrees based on the orientation of poled areas on the transducer 280.

In various embodiments, transducer modules can comprise one or more transduction elements. The transduction elements can comprise a piezoelectrically active material, such as lead zirconante titanate (PZT), or any other piezoelectrically active material, such as a piezoelectric ceramic, crystal, plastic, and/or composite materials, as well as lithium niobate, lead titanate, barium titanate, and/or lead metaniobate. In various embodiments, in addition to, or instead of, a piezoelectrically active material, transducer modules can comprise any other materials adapted to and/or configured for generating radiation and/or acoustical energy. In various embodiments, transducer modules can be adapted to and/or configured to operate at different frequencies and treatment depths. Transducer properties can be defined by an outer diameter (“OD”) and focal length (F_{L}). In one embodiment, a transducer can be adapted to and/or configured to have OD=19 mm and F_{L}=15 mm. In other embodiments, other suitable values of OD and F_{L} can be used, such as OD of less than about 19 mm, greater than about 19 mm, etc. and F_{L} of less than about 15 mm, greater than about 15 mm, etc. Transducer modules can be adapted to and/or configured to apply ultrasonic energy at different target tissue depths. As described above, in several embodiments, transducer modules comprise movement mechanisms adapted to and/or configured to direct ultrasonic treatment in a linear or substantial liner sequence of individual TCPs with a treatment spacing between individual TCPs. For example, treatment spacing can be about 1.0 mm, 1.1 mm, 1.2 mm, 1.3 mm, 1.4 mm, 1.5 mm, 1.6 mm, 1.7 mm, 1.8 mm, 1.9 mm, 2.0 mm, etc. In several embodiments, transducer modules can further comprise movement mechanisms adapted to and/or configured to direct ultrasonic treatment in a sequence so that TCPs are formed in linear or substantially linear sequences separated by a treatment spacing. For example, a transducer module can be adapted to and/or configured to form TCPs along a first linear sequence and a second linear sequence separated by treatment spacing between about 2 mm and 3 mm from the first linear sequence. In one embodiment, a user can manually move the transducer modules across the surface of a treatment area so that adjacent linear sequences of TCPs are created. In one embodiment, a movement mechanism can automatically move the transducer modules across the surface of a treatment area so that adjacent linear sequences of TCPs are created.

### Multifocal Zone Sequencing

In various embodiments, ultrasound imaging is used with a therapeutic tissue treatment. In various embodiments for improved ultrasound imaging, multiple focal zones are employed to obtain better signal quality and resolution through depth. For traditional, conventional diagnostic ultrasound scanners (linear, curvilinear, phased arrays, etc.), where the 2-D ultrasound images are formed without having to move the transducer, the sequence of acquiring these multiple focal zones are relatively inconsequential as precise placement of these focal zones can be controlled electronically. FIG. 3 illustrates a focal zone imaging that does not move while imaging, with an optionally electronically steered/translated aperture. For non-moving imaging transducers, focal zone positioning is precise, therefore focal zone sequencing is not employed. In traditional multiple focal zone imaging sequences, the order of the focal zone interrogation does vary. In various embodiments, a number “N” of focal zone sequence(s) will include 1, 2, 3, 4, 5, 6, 7, 8, 9, 10 or more focal zones. In one embodiment, N=1 for one focal zone. In one embodiment, N=4 for four focal zones. In one embodiment, N=8 for eight focal zones. In the following embodiments, N=4 is used, but any value N may be used in various embodiments. For example, with N=4, a 4-focal zone sequence will follow the progression (f1, f2, f3, f4) independent of location and direction of motion.

However, for moving imaging transducers (e.g., mechanically translated or steered arrays), this may become problematic, especially at increased speeds, due to the positional differences of the transducer as it scans through the multiple focal zones. This positional mis-registration is particularly magnified when forming imaging bidirectionally (forming both left-to-right and right-to-left images), as the region of interrogation between the two images might be different. This principle is demonstrated with a linearly translating circumstance, but the disclosure applies to all types of motion, including but not limited to translational, rotational, curved, two-dimensional and three-dimensional, or any combination thereof.

Embodiments of the imaging system disclosed herein address these misalignments. In instances, spatial mis-registration occurs due to the fact that the transducer is moving at one or more speeds while imaging. In particular, extreme focal zone can be placed apart between the two images, although they should be interrogating the same region of interest. When forming a 2-D image with a mechanically translated/steered transducer, the transmit/receive position of the transducer will vary, due to the fact that during the propagation time associated with an ultrasound signal, the transducer has also moved.

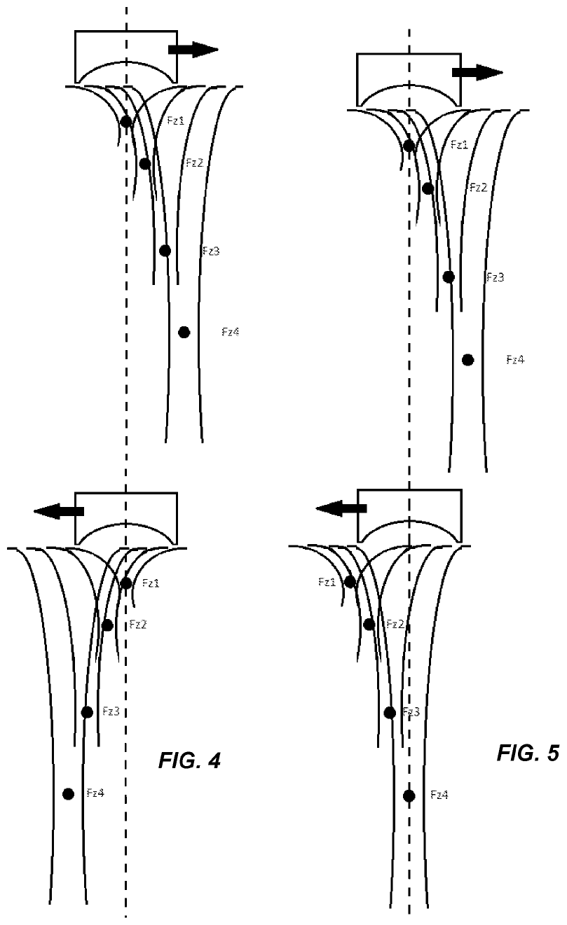

In one embodiment, a first direction-traveling (outbound) sequence shall proceed in order (f1, f2, f3, f4), and the second direction-traveling (returning) sequence is (f1, f2, f3, f4) or (f4, f3, f2, f1), thereby allowing better registration of two images. In one embodiment, a right-traveling (outbound) sequence shall proceed in order (f1, f2, f3, f4), and the left-traveling (returning) sequence is also (f1, f2, f3, f4), thereby allowing better registration of two images (FIG. 4). In one embodiment, an alternative sequence is proposed such that the right-traveling (outbound) sequence shall proceed in order (f1, f2, f3, f4), and the left-traveling (returning) sequence is reversed (f4, f3, f2, f1), thereby allowing better registration of two images (FIG. 5). In various embodiments, a direction can be left, right, forward, backward, up, down, clockwise or counterclockwise, and/or a combination of rotational and translation motions.

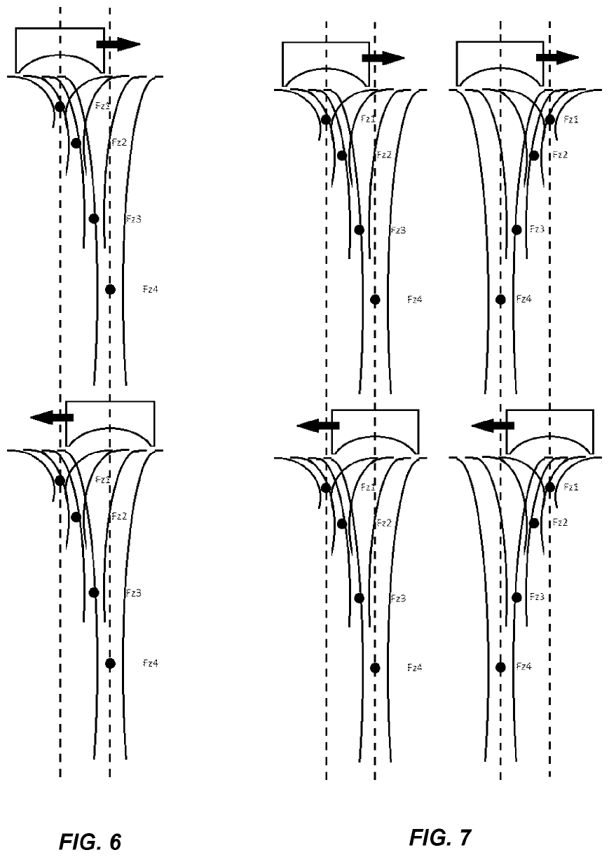

FIGS. 4-7 illustrate embodiments of directionally dependent focal zone sequencing. The left-traveling sequence can repeat or reverse order relative to the right-traveling sequence. As a result, the focal zone alignment has been improved. Further, the positions of acquisitions can be staggered, such that the same regions of interest are better registered between these two images. FIGS. 4-7 illustrate embodiments of a directionally dependent focal zone sequencing with different triggering locations. The spatial registration between right traveling and left traveling A-lines has been further improved by staggering the triggering locations. In an embodiment, an imaging system employs a novel sequence of two consecutive A-lines following progression of (line 1: f1, f2, f3, f4; line2: f1, f2, f3, f4) continuously. In an embodiment, an imaging system employs a novel sequence of two consecutive A-lines following progression of (line 1: f1, f2, f3, f4; line2: f4, f3, f2, f1) continuously. This sequence can be repeated across the entire field of view, and assuming an even number of vectors within the field of view, the returning sequence can have the exact same alternating pattern focal zone sequence, and the two images would be registered.

FIG. 7 illustrates an embodiment of a directionally dependent focal zone sequencing with sequences (f1-f2-f3-f4) and (f1-f2-f3-f4) or alternating between (f1-f2-f3-f4) and (f4-f3-f2-f1) on consecutive A-lines. In one embodiment, the entire field of view is spanned by an even number of A-lines and the left-traveling and right-traveling focal sequences are the same. Triggering locations still vary between the two images. In various embodiments, the multifocal zone imaging provides advantages for better correlation between first direction-traveling and second direction-traveling formed images. In various embodiments, the multifocal zone imaging provides advantages for improved effectiveness of B-mode imaging at faster (e.g., 2×, 3×, 4×) the scanning rate. In various embodiments, multifocal zone imaging is applied to any number of focal zones greater than one. In various embodiments, the number of focal zones is two, three, four, five, six, seven, eight, nine, ten, or more.

According to various embodiments, an ultrasound treatment system creates one, two or more simultaneous therapeutic treatment points and/or focal zones under the skin surface for a cosmetic treatment. The acoustic beam movement can be side-to-side, up-down, and/or at an angle. In one embodiment of mechanical dithering, the movement of the motion mechanism is sufficiently fast enough to create a flatter temperature profile around the intended TCP which either allows a reduction of total acoustic energy for the same effected tissue volume or the same total acoustic energy for a larger effected tissue volume or any combination thereof. In accordance with various embodiments, frequency modulation modifies the location of a focal zone and/or spacing between the focal zones, such that electronic dithering of beam via modulation of the frequency precisely alters and/or moves the position of the beam focus point(s). For example, in one embodiment, a spacing of 1.5 mm can be dithered with +/−0.1 mm using a small frequency swing. In various embodiments, any one or more spacings of 0.5, 0.75, 1.0, 1.2, 1.5, 2.0 mm can be dithered with +/−0.01, 0.05, 0.1, 0.12, 0.15, 0.20, 0.25, 0.30 mm using a frequency swing. In various embodiments, a frequency is modulated by 1-200% (e.g., 1%, 5%, 10%, 15%, 20%, 25%, 30%, 35%, 40%, 45%, 50%. 100%, 120%, 150%, 180%, 200% and any range therein).

In accordance with various embodiments, a cosmetic ultrasound treatment system and/or method can non-invasively produce single or multiple dithered cosmetic treatment zones and/or thermal coagulation points where ultrasound is focused in one or more locations in a region of treatment in tissue under a skin surface, and moved via changes in frequency (e.g., via frequency modulation). Some systems and methods provide cosmetic treatment at different locations in tissue, such as at different depths, heights, widths, and/or positions. In one embodiment, a method and system comprise a multiple depth/height/width transducer system configured for providing ultrasound treatment to one or more region of interest, such as between at least one depth of treatment region of interest, a superficial region of interest, and/or a subcutaneous region of interest. In one embodiment, a method and system comprise a transducer system configured for providing ultrasound treatment to more than one region of interest, such as between at least two points in various locations (e.g. at a fixed or variable depth, height, width, and/or orientation, etc.) in a region of interest in tissue. Some embodiments can split a beam to focus at two, three, four, or more focal points (e.g., multiple focal points, multi-focal points) for cosmetic treatment zones and/or for imaging in a region of interest in tissue. Position and/or dithering of the focal points can be positioned axially, laterally, or otherwise within the tissue. Some embodiments can be configured for spatial control, such as by the location and/or dithering of a focus point, changing the distance from a transducer to a reflecting surface, and/or changing the angles of energy focused or unfocused to the region of interest, and/or configured for temporal control, such as by controlling changes in the frequency, drive amplitude and timing of the transducer. In some embodiments the position and/or dithering of multiple treatment zones or focal points is achieved with poling, phasic poling, biphasic poling, and/or multi-phasic poling. In some embodiments the position of multiple treatment zones or focal points with phasing, such as in one embodiment, electrical phasing. As a result, changes in the location of the treatment region, the number, shape, size and/or volume of treatment zones or lesions in a region of interest, as well as the thermal conditions, can be dynamically controlled over time.

In accordance with various embodiments, a cosmetic ultrasound treatment system and/or method can create multiple cosmetic treatment zones using one or more of frequency modulation, phase modulation, poling, nonlinear acoustics, and/or Fourier transforms to create any spatial periodic pattern with one or multiple ultrasound portions. In one embodiment, a system simultaneously or sequentially delivers single or multiple treatment zones using poling at a ceramic level. In one embodiment, a poling pattern is function of focal depth and frequency, and the use of odd or even functions. In one embodiment, a poling pattern, which can be a combination of odd or even functions, is applied, and based on focal depth and/or frequency. In one embodiment, a process can be used in two or more dimensions to create any spatial periodic pattern. In one embodiment, an ultrasound beam is split axially and laterally to significantly reduce treatment time through the use of nonlinear acoustics and Fourier transforms. In one embodiment, modulation from a system and amplitude modulation from a ceramic or a transducer can be used to place multiple treatments zones in tissue, either sequentially or simultaneously.

In one embodiment, an aesthetic imaging and treatment system includes an ultrasonic probe that includes an ultrasound transducer configured to apply ultrasonic therapy to tissue at a plurality of locations at a focal depth with electronic dithering of multiple energy beam apertures with frequency modulation. In one embodiment, the system includes a control module coupled to the ultrasonic probe for controlling the ultrasound transducer.

In one embodiment, the system includes dithering configured to provide variable spacing between a plurality of individual cosmetic treatment zones. In one embodiment, a sequence of individual cosmetic treatment zones has a treatment spacing in a range from about 0.01 mm to about 25 mm (e.g., 1 mm, 1.5 mm, 2 mm, 2.5 mm, 3 mm, 5 mm, 10 mm, 20 mm and any value ranges therein), with a dithering alteration of the spacing by 1-50% (e.g., 1%, 5%, 10%, 15%, 20%, 25%, 30%, 35%, 40%, 45%, 50% and any range therein). In one embodiment, a sequence of individual cosmetic treatment zones has a treatment spacing in a range from about 0.01 mm to about 100 mm (e.g., 1 mm, 1.5 mm, 2 mm, 2.5 mm, 3 mm, 5 mm, 10 mm, 20 mm, 25 mm, 30 mm, 35 mm, 40 mm, 45, mm, 50 mm, 60 mm, 70 mm, 80 mm, 90 mm, and 100 mm, and any value ranges therein), with a dithering alteration of the spacing by 1-50% (e.g., 1%, 5%, 10%, 15%, 20%, 25%, 30%, 35%, 40%, 45%, 50% and any range therein).

In one embodiment, the system further includes a movement mechanism configured to be programmed to provide constant or variable spacing between the plurality of individual cosmetic treatment zones. In one embodiment, a sequence of individual cosmetic treatment zones has a treatment spacing in a range from about 0.01 mm to about 25 mm (e.g., 0.1, 0.5, 1, 2, 3, 4, 5, 6, 7, 8, 9, 10, 15, 19 mm or any range or value therein). In one embodiment, a sequence of individual cosmetic treatment zones has a treatment spacing in a range from about 0.01 mm to about 100 mm (e.g., 0.1, 0.5, 1, 2, 3, 4, 5, 6, 7, 8, 9, 10, 15, 20, 25, 30, 35, 40, 50, 100 mm or any range or value therein). In one embodiment, treatment zones are provided along a distance of about 25 mm. In one embodiment, treatment zones are provided along a distance of about 50 mm. In various embodiments, treatment zones are provided along a distance of 5 mm to 100 mm (e.g., 10 mm, 20 mm, 25 mm, 35 mm, 50 mm, 75 mm, 100 mm, and any amounts or ranges therein. In various embodiments, treatment zones are provided along a linear and/or curved distance.

For example, in some non-limiting embodiments transducers can be configured for a tissue depth of 0.5 mm, 1.0 mm, 1.5 mm, 2 mm, 3 mm, 4.5 mm, 6 mm, less than 3 mm, between 0.5 mm and 5 mm, between 1.5 mm and 4.5 mm, more than more than 4.5 mm, more than 6 mm, and anywhere in the ranges of 0.1 mm-3 mm, 0.1 mm-4.5 mm, 0.1 mm-25 mm, 0.1 mm-100 mm, and any depths therein (e.g., 6 mm, 10 mm, 13 mm, 15 mm). In several embodiments, tissue is treated at a depth below a skin surface and the skin surface is not impaired. Instead, the therapeutic effect achieved at the depth below the skin surface results in a favorable cosmetic appearance of the skin surface. In other embodiments, the skin surface is treated with ultrasound (e.g., at a depth less than 0.5 mm).

One benefit of a motion mechanism is that it can provide for a more efficient, accurate and precise use of an ultrasound transducer, for imaging and/or therapy purposes. One advantage this type of motion mechanism has over conventional fixed arrays of multiple transducers fixed in space in a housing is that the fixed arrays are a fixed distance apart. In one embodiment, the transducer module is configured to provide an acoustic power of the ultrasonic therapy in a range of between about 1 W to about 100 W (e.g., 3-30 W, 7-30 W, 21-33 W) and a frequency of about 1 MHz to about 10 MHz to thermally heat the tissue to cause coagulation. In one embodiment, the transducer module is configured to provide an acoustic power of the ultrasonic therapy in a range of between about 1 W to about 500 W for peak or average energy, (e.g., 3-30 W, 7-30 W, 21-33 W, 100 W, 220 W, or more) and a frequency of about 1 MHz to about 10 MHz to thermally heat the tissue to cause coagulation. In some embodiments, an instantaneous energy is delivered. In some embodiments, an average energy is delivered. In one embodiment, the acoustic power can be from a range of 1 W to about 100 W in a frequency range from about 1 MHz to about 12 MHz (e.g., 1 MHz, 3 MHz, 4 MHz, 4.5 MHz, 7 MHz, 10 MHz, 2-12 MHz), or from about 10 W to about 50 W at a frequency range from about 3 MHz to about 8 MHz (e.g., 3 MHz, 4 MHz, 4.5 MHz, 7 MHz). In one embodiment, the acoustic power can be from a range of 1 W to about 500 W in a frequency range from about 1 MHz to about 12 MHz (e.g., 1 MHz, 4 MHz, 7 MHz, 10 MHz, 2-12 MHz), or from about 10 W to about 220 W at a frequency range from about 3 MHz to about 8 MHz, or 3 MHz to 10 MHz. In one embodiment, the acoustic power and frequencies are about 40 W at about 4.3 MHz and about 30 W at about 7.5 MHz. An acoustic energy produced by this acoustic power can be between about 0.01 joule (“J”) to about 10 J or about 2 J to about 5 J. An acoustic energy produced by this acoustic power can be between about 0.01 J to about 60,000 J (e.g., via bulk heating, for body shaping, submental fat, abdomen and/or flanks, arms, inner thigh, outer thigh, buttocks, abdominal laxity, cellulite), about 10 J or about 2 J to about 5 J. In one embodiment, the acoustic energy is in a range less than about 3 J. In various embodiments, a treatment power is 1 kW/cm² to 100 kW/cm², 15 kW/cm² to 75 kW/cm², 1 kW/cm² to 5 kW/cm², 500 W/cm² to 10 kW/cm², 3 kW/cm² to 10 kW/cm², 15 kW/cm² to 50 kW/cm², 20 kW/cm² to 40 kW/cm², and/or 15 kW/cm² to 35 kW/cm².

In several of the embodiments described herein, the procedure is entirely cosmetic and not a medical act. For example, in one embodiment, the methods described herein need not be performed by a doctor, but at a spa or other aesthetic institute. In some embodiments, a system can be used for the non-invasive cosmetic treatment of skin.

In various embodiments, the ultrasonic treatment is at least one of a face lift, a brow lift, a chin lift, an eye treatment, a wrinkle reduction, a decolletage improvement, a buttock lift, a scar reduction, a burn treatment, a skin tightening (e.g., an abdominal laxity treatment), a blood vessel reduction, a treatment of a sweat gland, a sun spot removal, a fat treatment, and a cellulite treatment.

In several embodiments, provided are systems and methods that successfully improve the ultrasound imaging of tissue while moving, such as when an imaging transducer is on a motion mechanism. In various embodiments, higher resolution is achieved. In various embodiments, better imaging signal quality is obtained. In various embodiments, ultrasound imaging is used with a therapeutic tissue treatment.

In various embodiments, an ultrasound treatment and imaging system configured for reducing imaging misalignment, including an ultrasonic probe comprising an ultrasound therapy transducer adapted to apply ultrasonic therapy to tissue, an ultrasound imaging transducer adapted for imaging the tissue, and a motion mechanism for moving the ultrasound imaging transducer in a first direction and a second direction. In an embodiment, the ultrasound imaging transducer is mechanically attached to the motion mechanism. In an embodiment, the first direction is linear. In an embodiment, the second direction is linear. In an embodiment, the first direction is parallel to the second direction. In an embodiment, the first direction is opposite the second direction. In an embodiment, the ultrasound imaging transducer images with a first focal zone sequence order (e.g., f1, f2, . . . fN) when travelling in the first direction, the ultrasound imaging transducer images with a second focal zone sequence order (e.g., f1, f2, . . . fN; or fN, . . . f2, f1) when travelling in the second direction, and a spatial registration between the first direction imaging and the second direction imaging is improved by staggering a triggering location. In an embodiment, a control module is coupled to the ultrasonic probe for controlling the ultrasound imaging transducer.

In various embodiments, an ultrasound treatment and imaging system configured for reducing imaging misalignment, includes an ultrasonic probe comprising an ultrasound therapy transducer adapted to apply ultrasonic therapy to tissue, an ultrasound imaging transducer adapted for imaging the tissue, and a motion mechanism for moving the ultrasound imaging transducer in a first direction and a second direction. In an embodiment, the ultrasound imaging transducer is mechanically attached to the motion mechanism, wherein the first direction is linear, wherein the second direction is linear, wherein the first direction is parallel to the second direction, wherein the first direction is opposite the second direction, wherein the ultrasound imaging transducer images with a first focal zone sequence order (f1, f2, f3, f4) when travelling in the first direction, wherein the ultrasound imaging transducer images with a second focal zone sequence order (f1, f2, f3, f4) or (f4, f3, f2, f1) when travelling in the second direction. In one embodiment, a spatial registration between the first direction imaging and the second direction imaging is improved by staggering a triggering location, wherein the imaging system employs a sequence of two consecutive A-lines following progression of (line 1: f1, f2, f3, f4; line2: f1, f2, f3, f4) continuously; and a control module coupled to the ultrasonic probe for controlling the ultrasound imaging transducer. In one embodiment, a spatial registration between the first direction imaging and the second direction imaging is improved by staggering a triggering location, wherein the imaging system employs a sequence of two consecutive A-lines following progression of (line 1: f1, f2, f3, f4; line2: f4, f3, f2, f1) continuously; and a control module coupled to the ultrasonic probe for controlling the ultrasound imaging transducer.

In various embodiments, an ultrasound treatment and imaging system configured for reducing imaging misalignment, includes an ultrasonic probe comprising an ultrasound therapy transducer adapted to apply ultrasonic therapy to tissue, an ultrasound imaging transducer adapted for imaging the tissue, and a motion mechanism for moving the ultrasound imaging transducer in a first direction and a second direction. In an embodiment, the ultrasound imaging transducer is mechanically attached to the motion mechanism. In an embodiment, the first direction is opposite the second direction. In an embodiment, the ultrasound imaging transducer images with a focal zone sequence order (f1, . . . , fN), where N>1 when travelling in the first direction. In an embodiment, the ultrasound imaging transducer images with a second focal zone sequence order (f1, . . . , fN) or (fN, . . . , f1) when travelling in the second direction. In an embodiment, a spatial registration between the first direction imaging and the second direction imaging is improved by staggering a triggering location. In an embodiment, the imaging system employs a directionally dependent focal zone sequencing repeating (f1- . . . -fN) and (f1- . . . -fN) and/or with alternating between (f1- . . . -fN) and (fN- . . . -f1) on consecutive A-lines; and a control module coupled to the ultrasonic probe for controlling the ultrasound imaging transducer.

In an embodiment, the first direction of motion of the transducer is any one or more of the group consisting of: linear, rotational, and curved. In an embodiment, the second direction is the reversed path of the first direction. In an embodiment, the first direction of motion occurs in multiple dimensions and the second direction is the reversed path of the first direction. In an embodiment, the ultrasound imaging transducer images with a first focal zone sequence order is specified as (f1, . . . , fN), where N>1 (e.g., N is 2, 3, 4, 5, 6, or more). In an embodiment, the ultrasound therapy transducer is configured for treatment of tissue at a first set of locations that is positioned within a first cosmetic treatment zone and a second set of locations that is positioned within a second cosmetic treatment zone, the first zone being different from the second zone. In an embodiment, the ultrasound therapy transducer is adapted to apply ultrasonic therapy using amplitude modulation whereby a plurality of portions of the ultrasound transducer are adapted to emit ultrasonic therapy at a plurality of amplitudes of acoustic intensity, wherein a first amplitude is different than a second amplitude. In an embodiment, at least one portion of the ultrasonic transducer is adapted to emit ultrasonic therapy at two or more amplitudes of acoustic intensity, and wherein the amplitude of ultrasonic therapy emitted by the at least one portion of the piezoelectric varies over time. In an embodiment, the ultrasound transducer comprises piezoelectric material and the plurality of portions of the ultrasound transducer are adapted to create a plurality of corresponding piezoelectric material variations in response to an electric field applied to the ultrasound transducer. In an embodiment, the plurality of piezoelectric material variations comprise at least one of expansion of the piezoelectric material and contraction of the piezoelectric material. In an embodiment, the ultrasound transducer is adapted to apply ultrasonic therapy via phase shifting whereby a plurality of portions of the ultrasound transducer are adapted to emit ultrasonic therapy at a plurality of phases of acoustic intensity, wherein a first phase is different than a second phase. In an embodiment, the plurality of phases comprises discrete phase values. In an embodiment, the ultrasound transducer is adapted to apply ultrasonic therapy using amplitude modulation whereby a plurality of portions of the ultrasound transducer are adapted to emit ultrasonic therapy at a plurality of amplitudes of acoustic intensity, wherein a first amplitude is different than a second amplitude; and apply ultrasonic therapy whereby a plurality of portions of the ultrasound transducer are adapted to emit ultrasonic therapy at a plurality of phases of acoustic intensity, wherein a first phase is different than a second phase. In various embodiments, the ultrasonic treatment is at least one of: a face lift, a brow lift, a chin lift, an eye treatment, a wrinkle reduction, a decolletage improvement, a buttock lift, a scar reduction, a burn treatment, a skin tightening (e.g., a laxity treatment), a blood vessel reduction, a treatment of a sweat gland, a sun spot removal, a fat treatment, a cellulite treatment, a vaginal rejuvenation, and an acne treatment.

In various embodiments, a method of reducing imaging misalignment in a moving ultrasound probe, including staggering a triggering location of a spatial registration between a first direction imaging and a second direction imaging with an ultrasonic probe, the ultrasound probe comprising an ultrasound therapy transducer adapted to apply ultrasonic therapy to tissue, an ultrasound imaging transducer adapted for imaging the tissue, and a motion mechanism for moving the ultrasound imaging transducer in a first direction and a second direction, wherein the ultrasound imaging transducer is mechanically attached to the motion mechanism, wherein the first direction is opposite the second direction, wherein the ultrasound imaging transducer images with a focal zone sequence order (f1, . . . , fN), with N>1, wherein the ultrasound imaging transducer images with a first focal zone sequence order (f1, fN) when travelling in the first direction, wherein the ultrasound imaging transducer images with a second focal zone sequence order (f1, . . . , fN) or (fN, . . . , f1) when travelling in the second direction.

In an embodiment, N=any one of the group consisting of: 2, 3, 4, 5, 6, 7, 8, 9, and 10. In an embodiment, N=2. In an embodiment, N=4. In an embodiment, N=6. In an embodiment, N=4. In various embodiments, the ultrasound treatment is at least one of a face lift, a brow lift, a chin lift, an eye treatment, a wrinkle reduction, a décolletage improvement, a buttock lift, a scar reduction, a burn treatment, a tattoo removal, a skin tightening (e.g., an abdominal laxity treatment), a vein removal, a vein reduction, a treatment on a sweat gland, a treatment of hyperhidrosis, a sun spot removal, a fat treatment, a vaginal rejuvenation, and an acne treatment.

Minimizing Imaging Artifacts from Acoustic Reflections

In various embodiments, systems and methods for ultrasound imaging of tissue are adapted for and/or configured to use one or more focal zones in the tissue for imaging. In one embodiment, one focal zone is used for imaging. In one embodiment, one focal zone is used for imaging without therapy. In one embodiment, one focal zone is used for imaging with therapy. In various embodiments, two, three, four, or more focal zones are used for imaging. In various embodiments, two, three, four, or more focal zones are used for imaging without therapy. In various embodiments, two, three, four, or more focal zones are used for imaging with therapy. In various embodiments, an ultrasound transducer for imaging is placed directly in contact through acoustic coupling to a tissue such as a skin surface for imaging the one or more focal zones under the skin surface. In various embodiments, an ultrasound transducer for imaging has an offset gap between the imaging transducer and a portion of a housing (such as at an acoustic transmissive window, such as a PEEK window) in an ultrasound probe, whereby the portion of the housing is placed in contact through acoustic coupling to a tissue such as a skin surface for imaging the one or more focal zones under the skin surface. In some embodiments, an ultrasound transducer for imaging has an offset gap between the imaging transducer and a portion of a housing that uses two or more (e.g., 2, 3, 4, 5, 6, or more) focal zones can produce multipath artifacts from acoustic ultrasound energy that bounces between the imaging transducer and (i) the acoustic window and/or (ii) the region being imaged. These artifacts may obscure the clarity of the imaging.

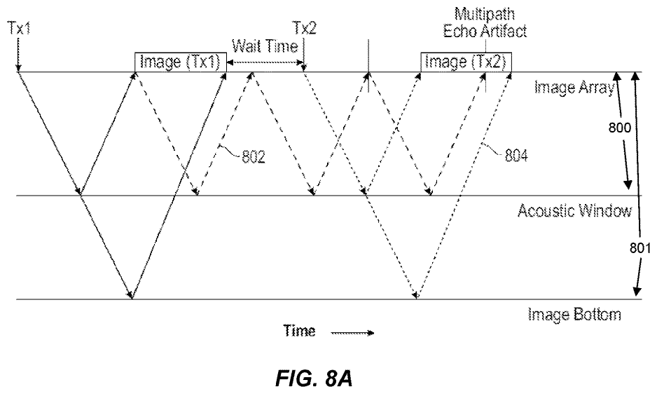

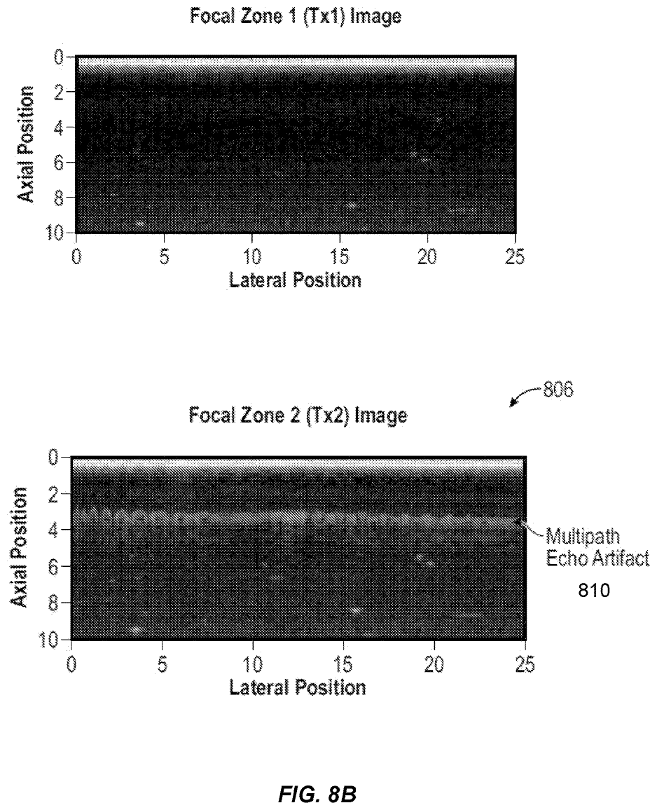

With references to FIGS. 8A and 8B, in some embodiments, a multipath artifact 810 may be produced when ultrasound energy transmits across an acoustic medium (e.g., an acoustic couplant, fluid, gel, liquid, such as water, glycerin, saline, and any combinations thereof) within a housing of an ultrasound imaging system. In some embodiments, artifacts are produced in an acoustic medium in an offset gap 800 between an imaging transducer (such as an imaging array) and a target issue. In some embodiments, this offset gap is 10.9, 11.1, 12.4 or 13.8 mm, but will also vary on the transducer temperature, amount of fluid within the transducer and the pressures (atmospheric or by the patient or clinician) exerted on the acoustic window. A multipath artifact 810 may be an ultrasound artifact in which an ultrasound beam reflects at an angle causing only a portion of the ultrasound beam to return to the transducer. This artifact can be produced from a portion of acoustic energy bouncing from being trapped inside the transducer housing between the imaging array and acoustic window. More specifically, the multipath artifact 810 may be produced from acoustic energy reflecting and repeatedly bouncing between the imaging array and the acoustic window. In one embodiment, these reflections may result in multipath artifacts 810 present at integer multiples of the distance between the imaging array and the acoustic window. The multipath artifacts 810 can blur and/or obscure the clarity of an image produced from the ultrasound imaging system and cause an ineffective or inefficient interpretation of the produced image.

In one embodiment, artifacts may be observed in successive imaging lines when executing B-mode imaging at high pulse repetition frequencies (“PRFs”), such as when acquiring multiple focal zones at depths (e.g., 0.1, 0.2, 0.3, 0.4, 0.5, 0.6, 0.7, 0.8, 0.9. 1.0, 1.1, 1.2, 1.3, 1.4, 1.5, 2, 2.5, 3, 3.5, 4, 4.5, 5, 5.5, 6, 6.5, 7, 8, 9, 10, 11, 12 mm below a skin surface and any ranges and values therein). This may produce a blurred or obscured image, or an image that can lead to an ineffective or inefficient interpretation of the produced image. In one embodiment, a multipath artifact 810 from an imaging transmitting at a given A-line/focal zone may present in the image data for a subsequent A-line/focal zone. Thus, the image produced from the partially reflected ultrasound beam(s) may also produce multipath artifacts 810 throughout the image, rather than in just one focal zone. This embodiment is illustrated schematically in FIG. 8A. As shown in FIG. 8A, the multipath artifact 810 may be present from a focal zone 1 transmit (Tx1) as a result of the ultrasound repeatedly bouncing between the imaging array and the acoustic window across an offset gap 800, and between the imaging array and the image bottom of region distance 801. This is represented as a Tx1 dashed line 802 shown in FIG. 8A. As shown in FIG. 8A, over time, the multipath artifact 810 continues to form from the overlapping ultrasound wave bouncing and reflections. When then sequencing a subsequent focal zone (Tx2) illustrated with dotted line 804, a reverberation of the multipath artifact 810 presents itself within the imaging data as well. FIG. 8A illustrates this Tx1 dashed line 802 present during the imaging of Tx2 dotted line 804 by showing the Tx1 dashed line 802 and Tx2 dotted line 804 intersecting or overlapping. As shown in FIG. 8B, the Focal Zone 2 (Tx2) image 806, the multipath artifact 810 is present and partially blurs/obscures the image. In some embodiments, the multipath artifact 810 may limit the imaging rates of the system because a wait time (or delay) may have to be set to a long enough period of time so that the multipath artifact echo is sufficiently attenuated. In various embodiments, this period of time can range between 30 to 60 microseconds (us) (e.g., 30-35, 30-40, 30-45, 30-50, 30-55, 35-40, 40-45, 45-50, 50-55, 55-60, 35-55, 35-50, 35-45, 40-50-, 40-55, 40-60, 45-55, 45-60, 50-60, 55-60 us, and values and ranges therein.

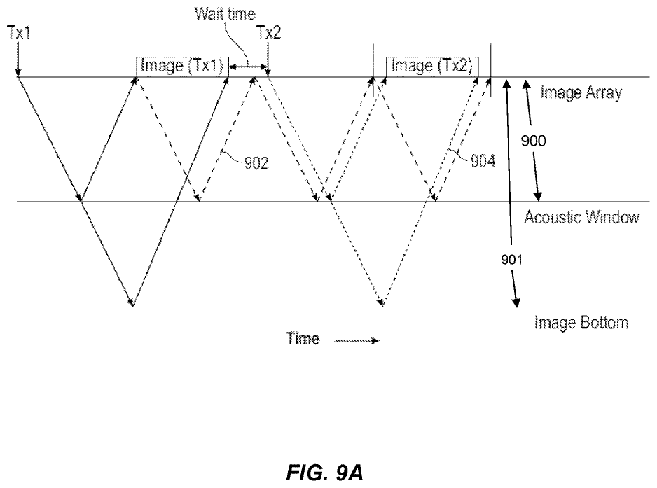

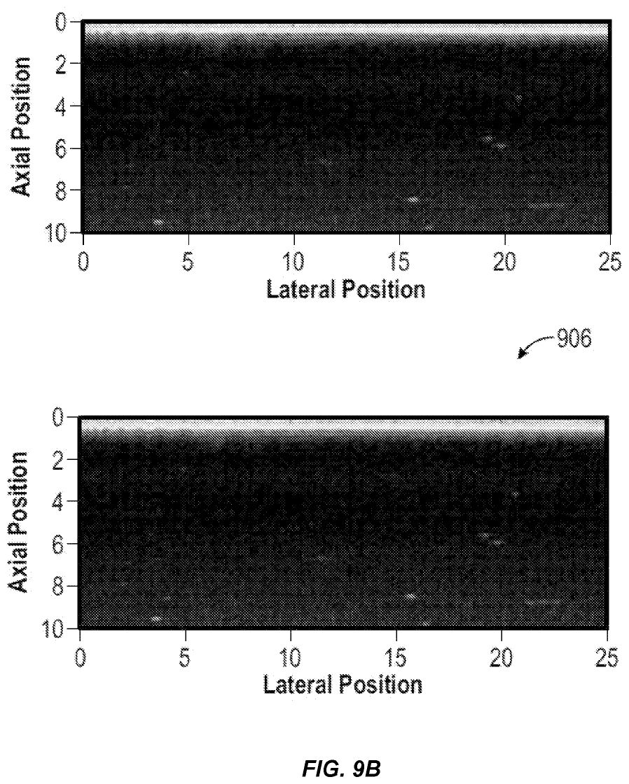

With reference to FIGS. 9A and 9B, for an imager-to-acoustic window offset gap distance 900 that remains static or constant, a wait time or Pulse Repetition Interval (PRI) may be strategically selected or calculated to reduce, or eliminate, a multipath artifact. For example, the wait time interval may be strategically selected such that the multipath artifacts (between the imaging array and the acoustic window across an offset gap 900, and between the imaging array and the bottom of an imaging region distance 901) present on subsequent focal zone image data are outside of the field of view of the transducer. As shown in FIG. 9A, the Tx1 dashed line 902 does not intersect the Tx2 904, but rather is parallel. In this embodiment, the multipath artifact 910 (not illustrated) is outside the field of view of the imaging. Furthermore, as shown in FIG. 9B, a multipath artifact 910 (not illustrated) echo is not present in a Tx2 image 906 produced, but instead is present outside of the image acquisition time for Tx2 904. In various embodiments, the static wait time can range from −30-60 microseconds (e.g., 30, 32, 32.5, 34, 36, 36.5, 37, 37.5, 38, 39, 39.5, 40, 42, 44, 44.5, 45, 45.5, 46, 48, 50, 52, 54, 56, 58, 60, and values and ranges therein)

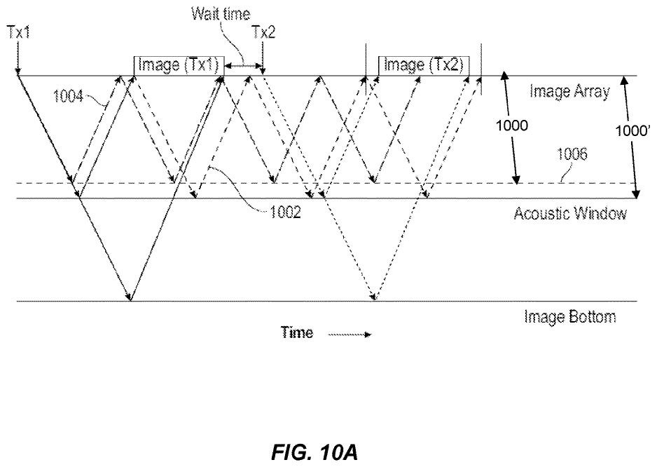

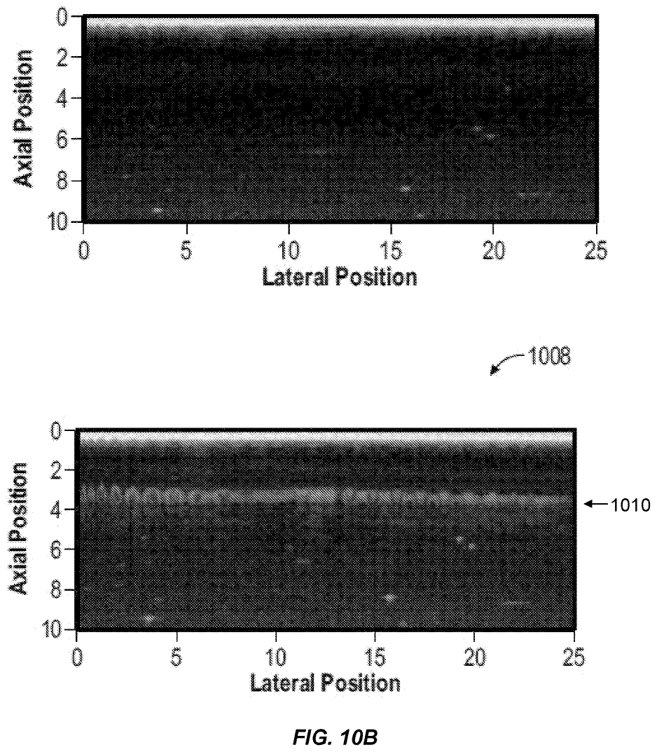

In some embodiments, the offset gap 1000 between the imaging transducer and the acoustic window varies (e.g., changes, is dynamic) between 1000-1000′. A dynamic offset gap 1000, 1000′ can change with variations in the temperature, pressure, and/or volume of coupling medium. The coupling medium temperature, pressure, and/or volume can change and fluctuate, and thereby deflect the acoustic window and change the offset gap distance 1000-1000′. In one embodiment, an ultrasound system housing may lose coupling medium via evaporation and/or leaking with use of the system over time. In one embodiment, an ultrasound system housing temperature of the coupling medium changes over time. In one embodiment, an ultrasound system housing pressure of the coupling medium changes over time. In one embodiment, the offset gap 1000, 1000′ to the acoustic window may be changed when a user or object presses against the acoustic window, therefore deflecting the acoustic window and changing the offset gap 1000, 1000′. As shown in an embodiment in FIG. 10A, the multipath artifact 1010 from Tx1 appears as a result of the sound repeatedly bouncing between a varying offset gap 1000-1000′ distance between the imaging array and the acoustic window. This is represented by Tx1 dashed line 1002.

Calculations to determine timing for reducing imaging artifacts for a dynamic offset are more complicated than for a static offset. With a static offset, the calculation for timing remains constant. However, with a dynamic offset, the calculation for timing changes. Using static calculations in a dynamic imaging environment will likely result in the appearance of imaging artifacts.

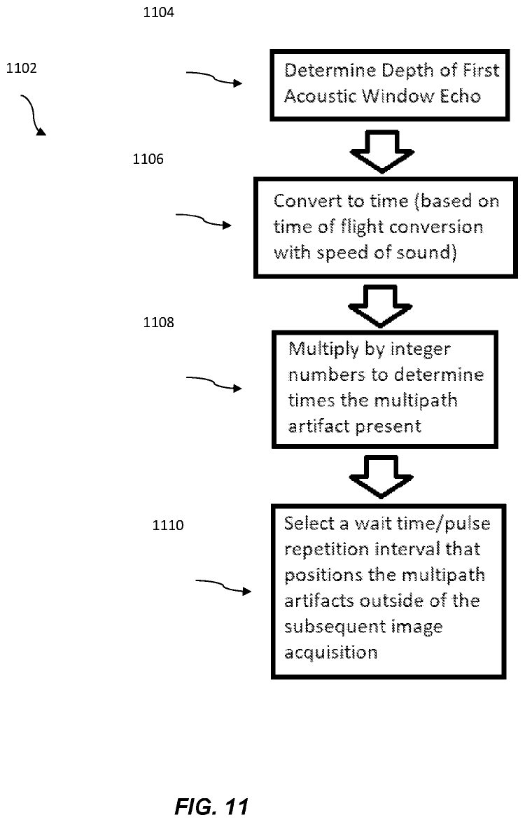

FIG. 11 illustrates a flow chart for dynamically setting an ultrasound imaging transmission wait time/pulse repetition interval (PRI) to reduce imaging multipath artifacts 810, 910, 1010 according to one embodiment. In one embodiment, a dynamic wait time calculation is implemented by extending the imaging region to include depths where the acoustic window may be located. With these additional depths, a dynamic offset distance 1000, 1000′ is measured within the B-mode image. The distance is measured by determining the offset depth of the first echo off the acoustic window. The speed of sound of the transducer coupling fluid at a given temperature is determined. The offset depth is calculated by converting a round-trip travel time. Subsequent multipath artifact timing is calculated by taking integer multiples of the round-trip travel time. In some embodiments, the speed of sound may be a constant value or be determined as a function of temperature if the internal coupling fluid temperature is also monitored. In some embodiments, the system may then dynamically set a wait time or a Pulse Repetition Interval such that the subsequent imaging transmit sequence is executed in which the multipath artifacts 1010 present at times outside of the received echo sampling interval of the subsequent transmission. In some embodiments, this calculation may be performed for each image frame. A-line, or focal zone transmit. In some embodiments, additionally, the focal zones may also be set on any of the intervals.

With further reference to FIG. 11, a method 1102 to dynamically set wait times or pulse repetition intervals is shown. At block 1104, the system determines the depth of the first acoustic window echo. This allows for the transducer to tailor the ultrasound image produced for the acoustic window actually being scanned. At block 1106, the system converts the determined depth to a time. The conversion is based on time of flight and the speed of sound in the acoustic medium. In one embodiment, at block 1108, the calculated time is multiplied by an integer to determine the number of times the multipath artifact may be present. At block 1110, a wait time or pulse repetition interval is selected. The selected wait time or pulse repetition interval may then position the multipath artifacts outside of the subsequent image acquisition. This dynamically sets the transmit wait time or pulse repetition interval and remove multipath echo artifacts.

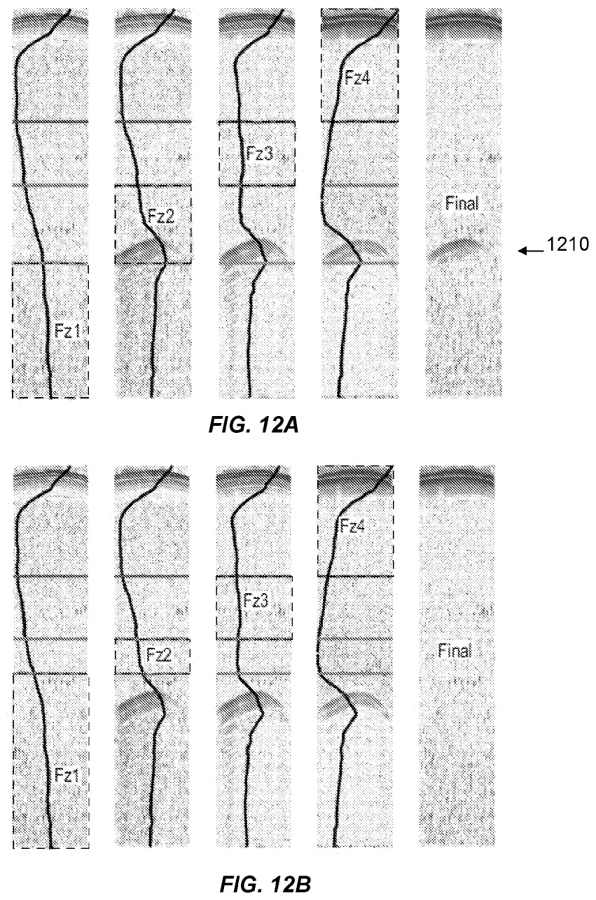

With reference to FIG. 12A and FIG. 12B, in some embodiments, a Pulse Repetition Interval (PRI, in units of time, e.g., 30-60 microseconds, e.g., 30, 32, 32.5, 34, 36, 36.5, 37, 37.5, 38, 39, 39.5, 40, 42, 44, 44.5, 45, 45.5, 46, 48, 50, 52, 54, 56, 58, 60, and values and ranges therein) is selected for imaging sequences utilizing multiple focal zone imaging. In one embodiment, a static PRI is implemented. In one embodiment, a dynamic PRI is implemented. In one embodiment, the multipath echo artifact 1210 is produced at a specific region inside the receive echo sampling interval for imaging sequences. In one embodiment, the multipath focal zone images are blended into a single image whereby the regions of the image that contain the artifact 1210 are not selected for display. This may be implemented when there is sufficient time between lateral locations for the multiple echo artifacts 1210 to subside. This calculation may be performed per image frame, A-line, or focal zone transmit. As shown in FIG. 12A, the image formed from the first focal zone transmit Fz1 does not contain an artifact 1210, but the subsequent focal zone images Fz2, for example, does contain the artifact 1210. However, when the focal zone images are blended, as shown in FIG. 12B, to form a single image, the first focal zone image Fz1 is used at the depths where the artifact 1210 is present in other focal zone images Fz2, Fz3, Fz4.

In various embodiments, 2, 3, 4, 5, 6, 7, 8 or more focal zones are employed. In some embodiments, as shown in FIG. 12A and FIG. 12B, four focal zones are employed Fz1, Fz2, Fz3, and Fz4. In one embodiment, the imaging sequence employs sufficient wait time between focal zone 4 from one lateral location to focal zone 1 for the subsequent lateral location. As a result, the multipath echo artifact only presents in focal zone 2 to focal zone 4 (Fz2, Fz3, Fz4). Regions of all four focal zone images, demarked with black dashed lines in FIGS. 12A and 12B, are blended and combined to form a single combined image. In one embodiment, blending regions are dynamically set, as shown in FIG. 12B, such that the multipath artifact does not present in the final image. As shown in FIG. 12B, the multipath artifact is effectively cropped out of the final image by altering the size of the four squares.

In one embodiment, calculating the depth where the multipath artifact presents within an image comprises the following steps:

Let do be the depth where the first echo of the acoustic window is detected within the B-mode image. Assuming constant velocity sound propagation, the time between the initial imaging transmit and arrival of this echo (to) is defined as:

$${t}_{0} = \frac{2}{{c}_{f} ( T )} , {d}_{0 ,}$$

Where c_{f} (T) is the speed of sound in the internal transducer fluid. This speed of sound value may be a constant or a function of temperature (T).

The times that multipath echo artifact arrive at the imaging array (t_{N}), therefore would occur at integer multiples of to:

$${t}_{N} = ( N + 1 ) \cdot {t}_{0} , N = 1 , 2 , 3 , …$$

If the displayed image axial field of view are defined at all depths d, where:

$${d}_{min} \leq d \leq {d}_{max}$$

And dₘᵢₙ and dₘₐₓ are the minimum and maximum depths of the displayed image, respectively, a dynamic time delay between successive imaging transmissions, t_{del}(N), may be selected such that two successive multipath echo times (t_{N} & t_{N+1}) are positioned outside of the axial field of view, such that:

$${t}_{del} ( N ) + \frac{2}{{c}_{f} ( T )} \cdot {d}_{min} < {t}_{N} ,$$

$${t}_{del} ( N ) + \frac{2}{{c}_{f} ( T )} \cdot {d}_{min} + \frac{2}{c} \cdot ( {d}_{max} - {d}_{min} ) > {t}_{N + 1}$$

Where c is defined as the speed of sound inside the target medium/patient.

Assigning t_{del} (N) to be:

$${t}_{del} ( N ) = {t}_{N + 1} - [ \frac{2}{{c}_{f} ( T )} \cdot {d}_{min} + k \cdot \frac{2}{c} \cdot ( {d}_{max} - {d}_{min} ) ] , 0 \leq k \leq 1$$

places the multipath echo at a relative depth, k, within the image. When k=0 the artifact is at the top of the image; when k=1, the artifact is at the bottom of the image.

Whether t_{del} (N) is static or dynamic, with its value known, the above equation may be rearranged to solve for k:

$$k = \frac{c \cdot [ {t}_{N + 1} - {t}_{del} ( N ) - \frac{2}{{c}_{f} ( T )} \cdot {d}_{min} ]}{2 \cdot ( {d}_{max} - {d}_{min} )}$$

After calculating k, the relative depth where multipath artifact presents within the image, focal zone blending depths may be dynamically selected to exclude the artifact from the final displayed image. For example, in one embodiment, the transducer fluid is water, at room temperature, c_{f}=1480 m/s, and when imaging into soft tissue, c=1540 m/s. If the first echo from acoustic window presents at 15 us, then the 4th echo would present at 60 us. With a static PRI of 36.5 us and the minimum and maximum imaging depths (from the imaging transducer) at 10.9 mm and 20.9 mm, then the relative depth (k) would be 0.68. Thus, the focal zone blending point could be selected such that the first focal zone would contain this relative depth, and therefore the artifact would not be included in the final, displayed image.

### Improving Imaging Alignment

In various embodiments, an imaging transducer may move with a motion mechanism 400 within a housing across a field of view at various speeds. In one embodiment, a motion mechanism 400 comprises a shaft, rod, screw, lead screw 401 for accurate and repeatable movement of an imaging transducer along a line, e.g., the imaging transducer is moved in and out, ingressing and egressing, along the shaft, rod, screw, lead screw 401. In various embodiments, a speed at which the imaging transducer may move across the field of view is 0.1-10.0 cycles per second (or hertz, Hz) (e.g., 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0 cycles per second including any values and ranges therein, e.g., 0.1-1.0, 0.1-2.0, 0.1-3.0, 0.1-4.0, 0.1-5.0, 1.0-2.0, 1.0-3.0, 1.0-4.0, 1.0-5.0, 2.0-3.0, 2.0-4.0, 2.0-5.0 cycles per second). In one embodiment, the imaging transducer moves across the field at a certain number of cycles per second. In one embodiment, B-mode images are acquired during both egress and ingress actions of the movement of the imaging transducer. Therefore, the frame rate may be increased, or doubled, to twice as many of the certain number of cycles or frames per second. For example, in one embodiment, the imaging transducer moves across the field of view at 3.0 cycles per second. In one embodiment, B-mode images are acquired during both egress and ingress actions of the movement of the imaging transducer and the frame rate is increased, or doubled, to 6.0 cycles or frames per second. However, in some embodiments, a slight misalignment of spatial interrogation between ingressing and egressing frames may be produced, resulting in inaccurate image that appears to be shaking. The misalignment may occur in more than one dimension (e.g., up-down, left-right, in-out, x-axis, y-axis, z-axis). Furthermore, in some embodiments, a misalignment may include a rotational component. In various embodiments, this imaging misalignment may occur in the lateral (e.g., left-right) and/or elevational (e.g., in-out) dimensions. In some embodiments, the result of this imaging misalignment may be that the image may appear to shake or may appear distorted, even if the imaged region is stationary or still.

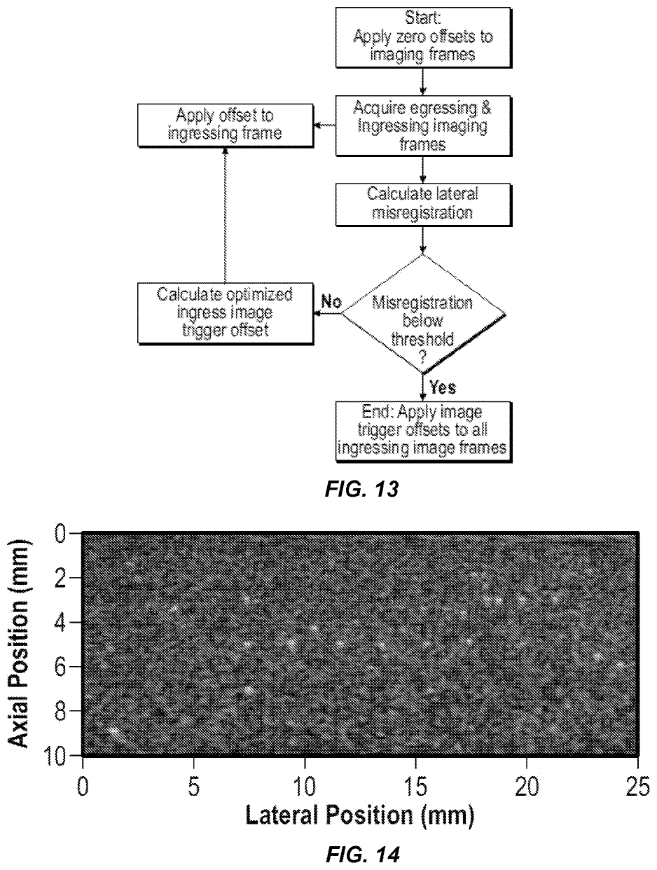

With reference to FIG. 13, in some embodiments, lateral imaging misalignments are reduced and/or eliminated through implementation of image trigger offsets. In some embodiments, elevational misalignments are addressed through implementation of at least one adaptive motion filter.

In some embodiments, lateral imaging misalignments are reduced or eliminated with egressing and ingression frames, imaging frames may be acquired first for both egress and ingress actions with minimal, or zero, offset between the two directions. In one embodiment, the image trigger locations for both frames may be identical. Subsequently, in some embodiments, a lateral cross correlation may then be performed on all ingression vectors. In one embodiment, an egressing frame may be used as a reference to determine which lateral location within the egressing frame best matches each ingression vector. Additionally, spatial interpolation may be utilized to match vectors to sub-pixel precision to better address any misalignments within the frames.

In some embodiments, the egressing image frame is taken as a reference image. In one embodiment, an imaging transducer moves with a therapy transducer that provides therapy in the egressing direction. In one embodiment, a guide marker within the displayed image indicates a location of a therapy dose.

In one embodiment, a lateral misregistration between the ingression vectors and the reference egressing image may be inverted and subsequently compiled to form a spatial image trigger offset curve for the next ingression frame acquisition. In one embodiment, the image trigger offset curve is not be physically realizable. This may occur when the image trigger offset causes the time difference between successive lateral location image acquisitions to be shorter than the minimum required imaging time at a single location causing the imaging acquisition data stream to overflow, and an error message is presented showing an imaging trigger fault. In order to address this, in one embodiment, a cost function is formulated in order to minimize the difference between the ideal acquisition delays and the realizable acquisition delays.

In some embodiments, a cost function is performed by seeding an absolute offset at each ingression location, in combination with applying physical limitations of the motion profile of the module and propagating realizable trigger offsets away from the absolute positions along the entire lateral range of travel for the module. This produces N realizable trigger offset curves, where N is the total number of lateral locations within the image. With an optimized, realizable ingression image trigger offset curve, a new set of imaging frames is acquired. The egressing frame remains unaltered; however, the realizable imaging trigger delays may be applied to the ingression imaging frame acquisition. Next, in some embodiments, the process may be repeated to calculate a new set of misregistration offsets and a further refined ingress image trigger delay curve. The process may be repeated until the two images converge and any lateral misregistration is suppressed to be below a specified predetermined threshold. In some embodiments, when the misregistration is below the threshold, the imaging trigger offsets may be programmed into the transducer so that all subsequent ingression images may be acquired with these offsets applied. In one embodiment, redundant realizable trigger offsets curves are eliminated. Where one curve crosses another, the two curves are mixed and matched and the cost function is used to remove suboptimal curves until a single optimized realizable trigger offset curve is obtained.

As shown in FIG. 13, in one embodiment of a method 1302 addresses imaging inaccuracies, shaking, and/or blurriness within images due to misalignment between ingression and egressing frames collected by a system to improve lateral registration. At block 1304, the system applies minimal or zero offsets to the imaging frames. At block 1306, egressing and ingression imaging frames are acquired by the system. At block 1308, the lateral misregistration is calculated by the system. At block 1310, the system determines whether the misregistration is below a predetermined threshold. At block 1312, if the misregistration is below the predetermined threshold, at least one image trigger offset is applied to all ingression image frames. However, at block 1314, if the misregistration is not below the predetermined threshold, the system calculates optimized ingress image trigger offsets. If the misregistration is not below the predetermined threshold, at block 1316, the system will then apply at least one image trigger offset to the ingression frame.

In some embodiments, elevational imaging misregistration is addressed after image acquisition with temporal filters that mitigate elevational misregistration artifacts. In one embodiment, one or more temporal filters are applied to B-mode images to eliminate or minimize elevational misregistration. Temporal filters may be applied by displaying an average of the previous N images, where N>1 (e.g., N=2, 3, 4, 5, 6, 7, 8, 9, 10, 25, 50, 100). This may be effective when imaging static targets and there is good spatial registration between the averaged frames. In some embodiments, however, when the transducer or the target is moving, a temporal filter may introduce a blurring effect as a result of imaging frames without lateral registration are being averaged together. In some embodiments, an adaptive temporal motion filter averages and/or stabilizes the B-mode images while the transducer is moving. In some embodiments, detecting motion may be performed with one or more sensors. Such sensors may include gyros or accelerometers. Additionally, in some embodiments, motion may be detected by the images themselves. In one embodiment, image correlation coefficients across multiple frames is calculated in real time.

In one embodiment, a temporal filter is activated (for a blending effect) when imaging correlation coefficients are optimized, and the temporal filter is deactivated when the coefficient drops below a certain level (to cease the blending effect).

In some embodiments, slight misalignments between successive frames (e.g., between first and second images, outgoing and incoming images) result in the correlation coefficient varying depending on the amount of misalignment. In one embodiment, at least two independent correlation coefficients are calculated to address this. In one embodiment, one coefficient is calculated using only outgoing images, while a second coefficient is calculated using only incoming images. This results in coefficients that are more stable and repeatable between imaging transducers, and the combination of the at least two coefficients may maintain, for example, at least a 6 frames per second calculations rate. In one embodiment, a temporal stabilization filter is engaged dependent on calculating the correlation coefficient with the current frame and the imaging frame and comparing the correlation coefficient with a threshold. In one embodiment, a correlation coefficient is calculated with the current frame and the imaging frame (e.g., 2, 4, 6 . . . ) frames ago, and comparing this coefficient with a threshold determines whether the temporal stabilization filter is engaged.

With reference to FIG. 14, imprecise imaging transducer positioning between inward and outward trajectories in a moving imaging device may cause image shaking and/or blurriness. In some embodiments, temporal motion artifact may be quantified. Using the raw quadrature detection (IQ) data, a correlation coefficient (“CC”) is calculated between any two frames (e.g., frames F & G).

$$F ( t ) = {I}_{1} ( t ) * cos ( ω * t ) + {Q}_{1} ( t ) * sin ( ω * t )$$

$$G ( t ) = {I}_{2} ( t ) * cos ( ω * t ) + {Q}_{2} ( t ) * sin ( ω * t )$$

$$CC ( t ) = \frac{Re \{ F ( t ) * G ( t ) \}}{\sqrt{‖ F ( t ) ‖ * ‖ G ( t ) ‖}} = \frac{( {I}_{1} ( t ) * {I}_{2} ( t ) ) + ( {Q}_{1} ( t ) * {Q}_{2} ( t ) )}{\sqrt{( {{I}_{1} ( t )}^{2} + {{Q}_{1} ( t )}^{2} ) * ( {{I}_{2} ( t )}^{2} + {{Q}_{2} ( t )}^{2} )}}$$

$$CC ( t ) = 1 ; when there is perfect correlation ( F ( t ) = G ( t ) )$$

$$CC ( t ) = 0 ; when there is no correlation$$

$$CC ( t ) = - 1 ; when there is perfectly inverted correlation$$

In some embodiments, these calculations provide for the performance of two-dimensional pattern matching to maximize the correlation coefficient and determine the location of each pixel in an image.

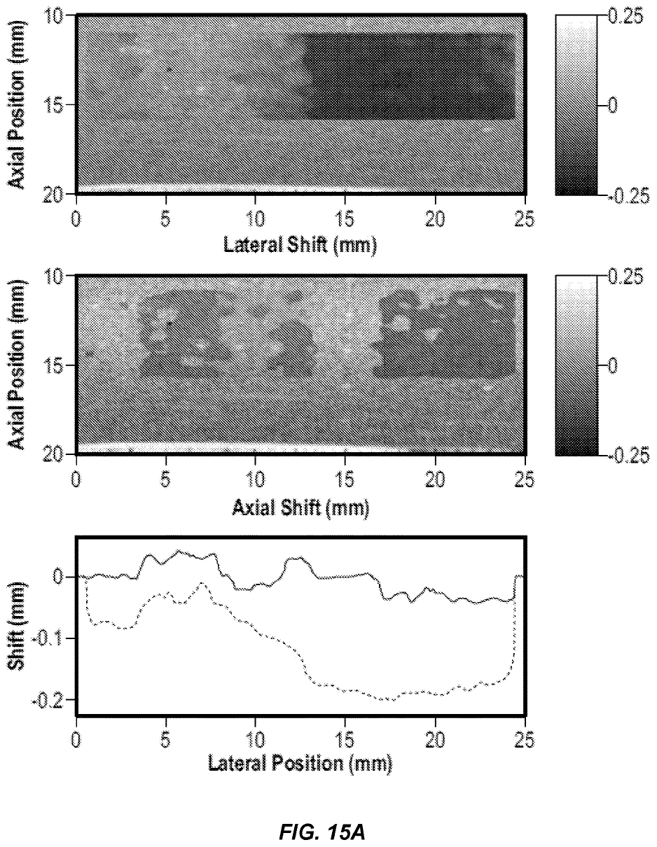

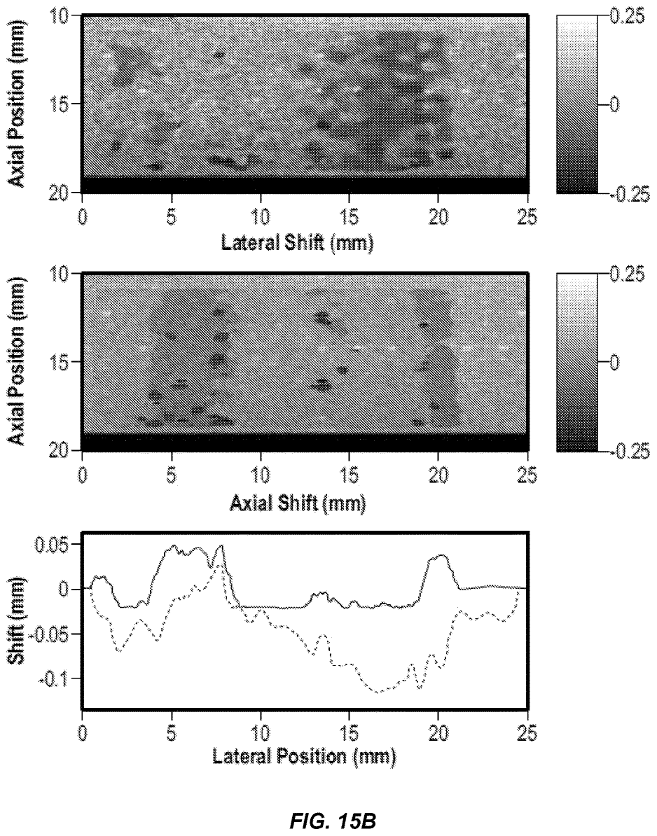

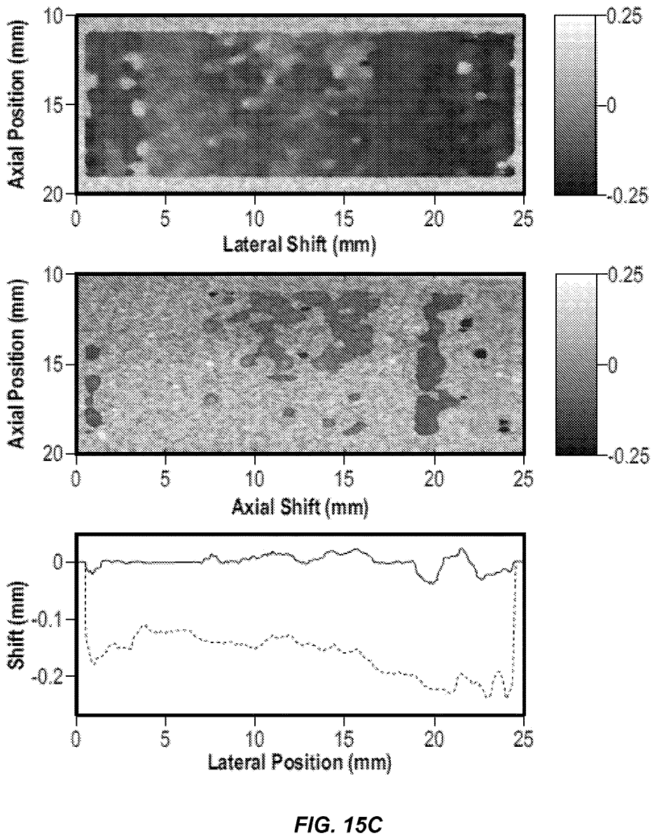

In one embodiment, mapping the temporal motion artifact as shown in FIG. 15A, has the temporal motion of the artifact appearing predominantly lateral. In one embodiment, mapping the temporal motion artifact as shown in FIG. 15B, has the temporal motion of the artifact appearing to be temporally stable. In one embodiment, mapping the temporal motion artifact as shown in FIG. 15C, has the temporal motion of the artifact appearing to be uniform in depth. In some embodiments, the quantification of the temporal motion artifact varies from transducer to transducer.

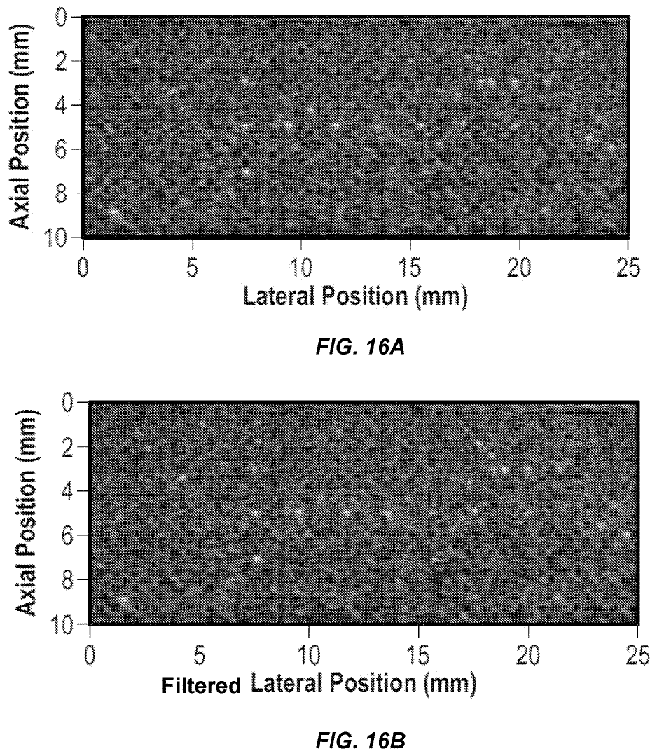

With reference to FIG. 16A and FIG. 16B, in one embodiment addressing imaging misregistration with only lateral shifts, a measurement of a specific shift in each imaging transducer is taken during the manufacture of that imaging transducer. With the measured shift value, the imaging system shifts the imaging data to the nearest pixel (e.g., nearest neighbor interpolation) based on the specific measured shift value. This method stabilizes images with exclusively lateral shifts—however, out of plane movement and subpixel decorrelation may not be addressed which may cause imaging shifting to persist. FIG. 16A illustrates an image with pixels shifting laterally in a back-and-forth, side-to-side movement. FIG. 16B illustrates the pixel alignment stabilized after application of the filter.

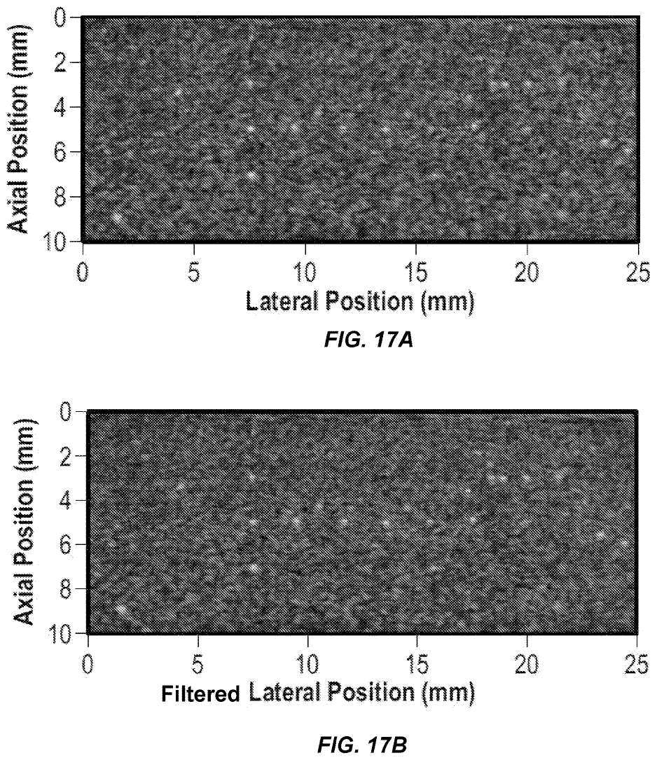

With reference to FIG. 17A and FIG. 17B, in one embodiment addressing imaging misregistration in elevation, consecutive imaging frames are temporally averaged to address lateral misregistration. In some embodiments, temporally averaging consecutive frames stabilize images. In some embodiments, temporally averaging consecutive frames can degrade speckle contract and image resolution. FIG. 17A illustrates an image with pixels shifting in an elevational direction. FIG. 17B illustrates the pixel alignment stabilized after application of the filter.

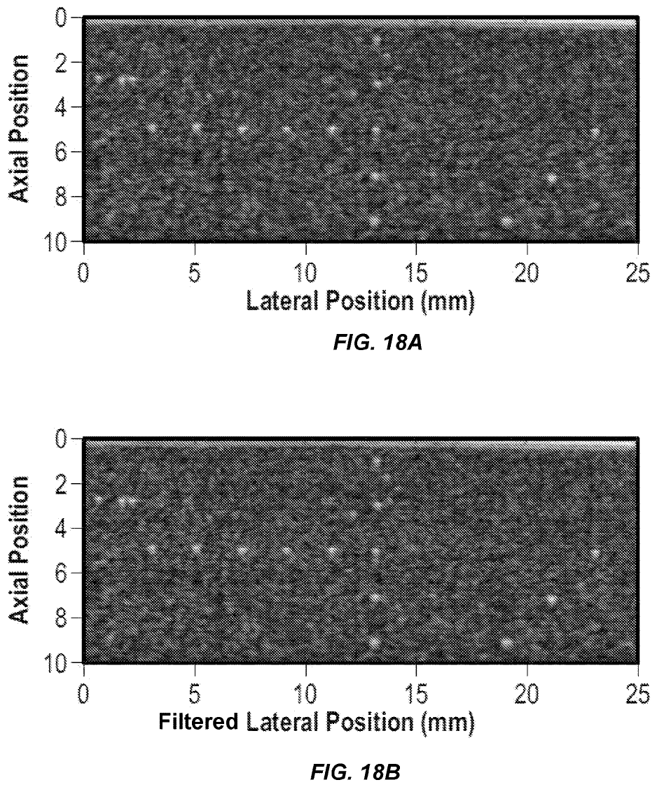

With reference to FIG. 18A and FIG. 18B, in one embodiment imaging misregistration and/or misalignment is reduced by both shifting data (as with the embodiment of FIGS. 16A and 16B) and temporally averaging consecutive frames (as with the embodiment of FIGS. 17A and 17B). The shifting data preserves imaging resolution and corrects large lateral motion artifacts (e.g., >1 pixel) that are consistently present. The temporally averaging of consecutive frames minimizes smaller motion artifacts (e.g., <1 pixel) in any direction.

In one embodiment, correlation coefficients increase when the image is stationary. In one embodiment, a correlation coefficient is less than 0.5. In one embodiment, a correlation coefficient may vary from imaging transducer to imaging transducer. In one embodiment, a correlation coefficient contrast marginally changes when shifted images are compared. In one embodiment, subpixel and out of plane decorrelation is present.

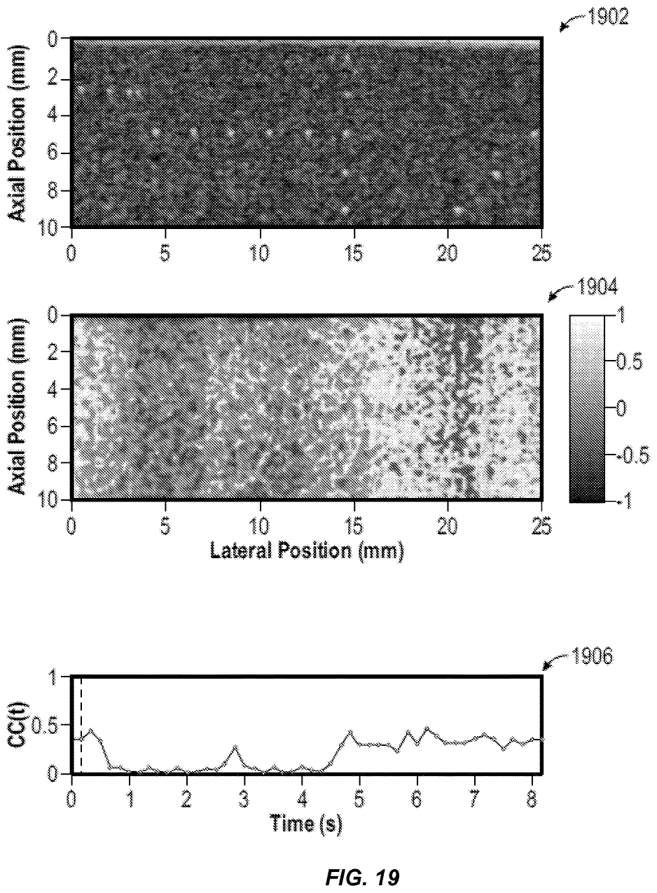

As shown in FIG. 19, in one embodiment, graph 1902 and graph 1904 show imaging pixels with lateral movement over time. In one embodiment, graph 1906 demonstrates the correlation coefficient changes over time.

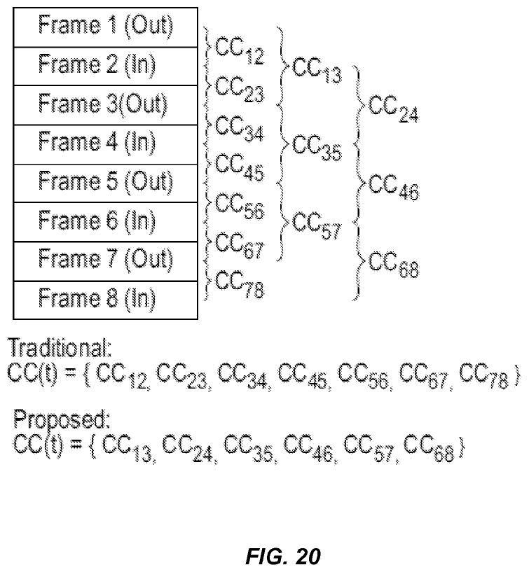

With reference to FIG. 20, in some embodiments, alternate frame correlation better reflects and accounts for the presence of motion while imaging. In one embodiment, this contributes to minimizing the loss in frame rate and/or update rate.

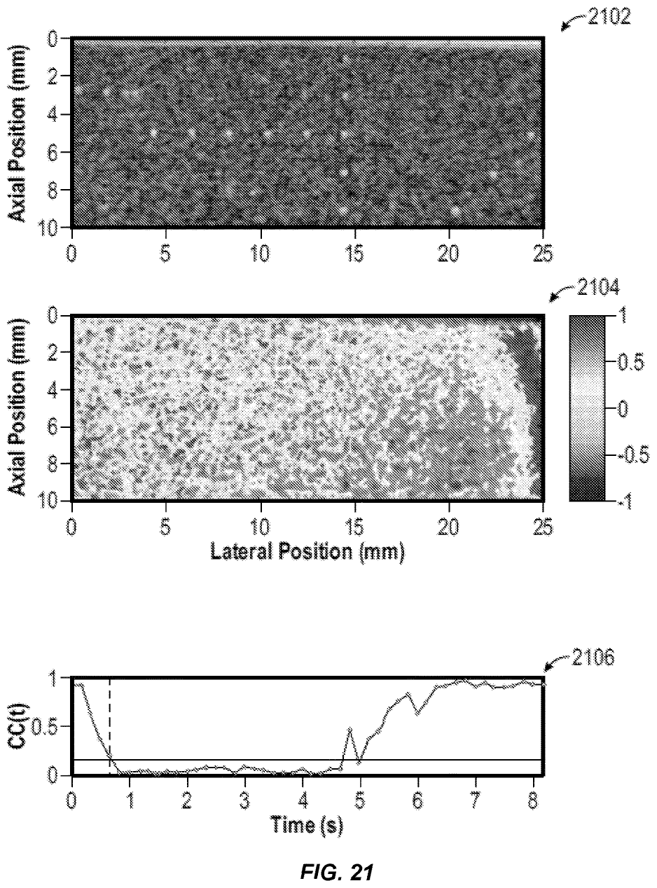

In some embodiments, with reference to FIG. 21, an imaging system comprises independently correlating outgoing and incoming images. In one embodiment, the correlation coefficient approaches 1 when the image is stationary and the correlation coefficient approaches 0 when the image is moving. In one embodiment, the correlation coefficient varies between 0-1, 0-0.5, 0-0.4, 0.0-0.3, 0-0.2, or 0-0.1. In various embodiments, a correlation coefficient varies between imaging transducers. Graph 2106 shows an embodiment of a correlation coefficient approaching 1 over time.

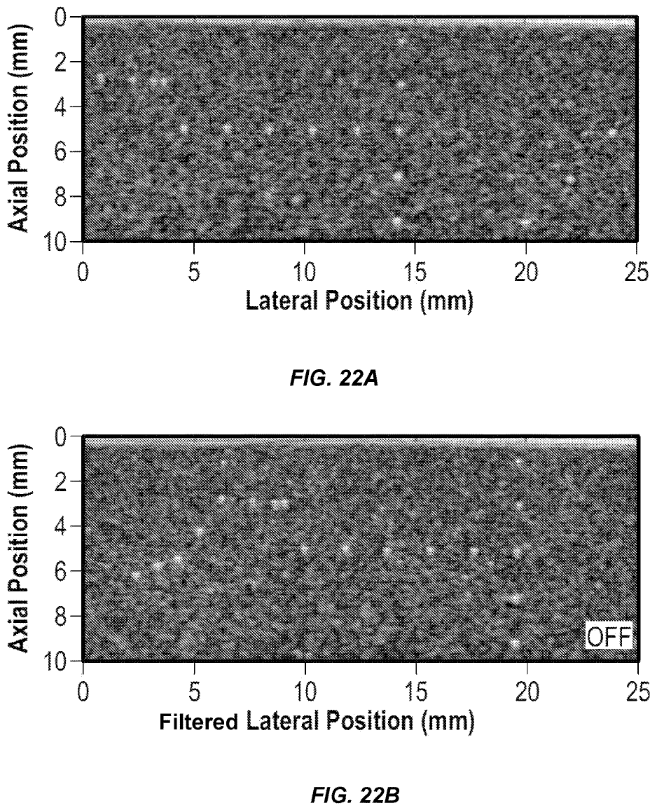

In some embodiments, with reference to FIG. 22A and FIG. 22B, an adaptive temporal motion filter with lateral misregistration correction corrects lateral misregistration when movement is sensed. In one embodiment, the temporal motion filter stabilizes imaging when the field of view is stationary. In one embodiment, the temporal motion filter is disable the when the field of view is moving, therapy preserving temporal resolution.

Some embodiments and the examples described herein are examples and not intended to be limiting in describing the full scope of compositions and methods of these invention. Equivalent changes, modifications and variations of some embodiments, materials, compositions and methods can be made within the scope, with substantially similar results.

While the embodiments here are capable of various modifications, and alternative forms, specific examples thereof have been shown in the drawings and are herein described in detail. It should be understood, however, that the embodiments are not to be limited to the particular forms or methods disclosed, but to the contrary, to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “coupling a transducer module with an ultrasonic probe” include “instructing the coupling of a transducer module with an ultrasonic probe.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 1 mm” includes “1 mm.”

What is claimed is:

1. An ultrasound imaging system configured for reducing imaging artifacts, comprising:
an ultrasonic probe comprising:
an ultrasound imaging transducer adapted for imaging a tissue region,
a housing comprising an acoustic window,
a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance,
an acoustic coupling medium within the housing configured to acoustically couple the ultrasound imaging transducer to the acoustic window,
a motion mechanism for moving the ultrasound imaging transducer in a first direction and a second direction,
wherein the ultrasound imaging transducer images with a focal zone sequence order (f₁, . . . , f_{N}), where N>2 when travelling in the first direction,
wherein the ultrasound imaging transducer images with a second focal zone sequence order (f₁, . . . , f_{N}) when travelling in the second direction; and
a control module coupled to the ultrasonic probe for controlling the ultrasound imaging transducer,
wherein the control module is configured to reduce at least one multipath echo artifact via a dynamically set pulse repetition interval.

2. The ultrasound imaging system of claim 1, wherein the dynamically set pulse repetition interval is further configured to:
measure the first offset depth;
calculate a first offset time based on the first offset depth;
multiply the first offset time by an integer to determine a presence of the at least one multipath echo artifact; and
select a pulse repetition interval configured to position the at least one multipath echo artifact outside of a displayed ultrasound image.

3. An ultrasound imaging system configured for reducing imaging artifacts, comprising:
an ultrasonic probe comprising:
an ultrasound imaging transducer adapted for imaging a tissue region,
a housing comprising an acoustic window,
a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance,
an acoustic coupling medium within the housing configured to acoustically couple the ultrasound imaging transducer to the acoustic window,
a motion mechanism for moving the ultrasound imaging transducer in a first direction and a second direction,
wherein the ultrasound imaging transducer images with a focal zone sequence order (f₁, . . . , f_{N}), where N>2 when travelling in the first direction,
wherein the ultrasound imaging transducer images with a second focal zone sequence order (f₁, . . . , f_{N}) when travelling in the second direction; and
a control module coupled to the ultrasonic probe for controlling the ultrasound imaging transducer,
wherein the control module is configured to reduce at least one multipath echo artifact via a dynamically set one or more focal zone blend points.

4. The ultrasound imaging system of claim 3, wherein the at least one dynamically set focal zone blend point is further configured to:
measure the first offset depth;
calculate a first offset time based on the first offset depth;
multiply the first offset time by an integer to determine a presence of the at least one multipath echo artifact; and
select at least one focal zone blend point configured to position the at least one multipath echo artifact outside of a displayed ultrasound image.

5. The ultrasound imaging system of any one of claims 1-4, wherein the dynamic offset distance varies based on a changing volume of the acoustic coupling medium, wherein the changing volume of the acoustic coupling medium is a result of evaporation or leaking of the acoustic coupling medium from the housing.

6. The ultrasound imaging system of any one of claims 1-4, wherein the dynamic offset distance varies based on a changing temperature of the acoustic coupling medium.

7. The ultrasound imaging system of any of the preceding claims, wherein the dynamic offset distance varies based on a changing pressure of the acoustic coupling medium.

8. The ultrasound imaging system of any one of claims 1-4, wherein the dynamic offset distance varies with a speed of the motion mechanism in at least one of the first direction and the second direction.

9. The ultrasound imaging system of any one of claims 1-4, further comprising a therapy transducer configured to apply ultrasonic therapy to the tissue.

10. The ultrasound imaging system of any one of claims 1-4, wherein N=any one of 2, 3, or 4.

11. An ultrasound imaging system configured for reducing imaging artifacts, comprising:
an ultrasonic probe comprising:
an ultrasound imaging transducer adapted for imaging a tissue region,
a housing comprising an acoustic window,
a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance,
means for moving the ultrasound imaging transducer in a first direction and a second direction, and
a control module coupled to the ultrasonic probe for controlling the ultrasound imaging transducer,
wherein the control module is configured to reduce at least one multipath echo artifact via a dynamically set pulse repetition interval.

12. An ultrasound imaging module configured for reducing imaging artifacts, comprising:
an ultrasound imaging transducer adapted for imaging a tissue region,
a housing comprising an acoustic window,
a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance,
means for moving the ultrasound imaging transducer in a first direction and a second direction, and
a control module coupled to the ultrasonic probe for controlling the ultrasound imaging transducer,
wherein the control module is configured to reduce at least one multipath echo artifact via a dynamically set pulse repetition interval.

13. The ultrasound imaging module of claim 12, wherein the at least one dynamically set focal zone blend points is further configured to:
measure the first offset depth;
calculate a first offset time based on the first offset depth;
multiply the first offset time by an integer to determine a presence of the at least one multipath echo artifact; and
select a at least one focal zone blend point configured to position the at least one multipath echo artifact outside of a displayed ultrasound image.

14. An ultrasound imaging device configured for reducing imaging artifacts, comprising:
an ultrasonic module comprising:
an ultrasound imaging transducer adapted for imaging a tissue region,
a housing comprising an acoustic window,
a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance,
means for moving the ultrasound imaging transducer in a first direction and a second direction, and
a control module coupled to the ultrasonic probe for controlling the ultrasound imaging transducer,
wherein the control module is configured to reduce at least one multipath echo artifact via a dynamically set pulse repetition interval.

15. The ultrasound imaging device of claim 14, wherein the dynamically set at least one focal zone blend point is further configured to:
measure the first offset depth;
calculate a first offset time based on the first offset depth;
multiply the first offset time by an integer to determine a presence of the at least one multipath echo artifact; and
select at least one focal zone blend point configured to position the at least one multipath echo artifact outside of a produced ultrasound image.

16. The ultrasound imaging device of any one of claims 14-15, wherein the dynamic offset distance varies based on a changing volume of the acoustic coupling medium, wherein the changing volume of the acoustic coupling medium is a result of evaporation or leaking of the acoustic coupling medium from the housing.

17. The ultrasound imaging device of any one of claims 14-15, wherein the dynamic offset distance varies based on a changing temperature of the acoustic coupling medium.

18. The ultrasound imaging device of any one of claims 14-15, wherein the dynamic offset distance varies based on a changing pressure of the acoustic coupling medium.

19. The ultrasound imaging device of any one of claims 14-15, wherein the dynamic offset distance varies with a speed of the mechanism in at least one of the first direction and the second direction.

20. The ultrasound imaging device of any one of claims 14-15, further comprising a therapy transducer configured to apply ultrasonic therapy to the tissue.

21. The ultrasound imaging device of any one of claims 14-15, wherein N=any one of 2, 3, or 4.

22. A method of reducing multipath echo artifacts from an ultrasound image, comprising:
providing an ultrasonic probe comprising:
an ultrasound imaging transducer adapted for imaging a tissue region,
a housing comprising an acoustic window,
a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance,
an acoustic coupling medium within the housing configured to acoustically couple the ultrasound imaging transducer to the acoustic window,
a motion mechanism for moving the ultrasound imaging transducer in a first direction and a second direction,
wherein the ultrasound imaging transducer images with a focal zone sequence order (f₁, . . . , f_{N}), where N>2 when travelling in the first direction,
wherein the ultrasound imaging transducer images with a second focal zone sequence order (f₁, . . . , f_{N}) when travelling in the second direction; and
measuring a first offset depth;
calculating a first offset time based on the first offset depth;
multiplying the first offset time by an integer to determine a presence of the at least one multipath echo artifact; and
selecting a pulse repetition interval configured to position the at least one multipath echo artifact outside of a displayed ultrasound image.

23. A method of reducing multipath echo artifacts from an ultrasound image, comprising:
providing an ultrasonic probe comprising:
an ultrasound imaging transducer adapted for imaging a tissue region,
a housing comprising an acoustic window,
a dynamic offset distance between the ultrasound imaging transducer and the acoustic window, wherein the dynamic offset distance changes over time, wherein the dynamic offset distance comprises a first offset distance and a second offset distance, wherein the first offset distance is different than the second offset distance,
an acoustic coupling medium within the housing configured to acoustically couple the ultrasound imaging transducer to the acoustic window,
a motion mechanism for moving the ultrasound imaging transducer in a first direction and a second direction;
calculating a first offset time based on the first offset depth;
multiplying the first offset time by an integer to determine a presence of the at least one multipath echo artifact; and
selecting at least one focal zone blend point configured to position the at least one multipath echo artifact outside of a displayed ultrasound image.

24. The method of any one of claims 22-23, further comprising:
imaging a tissue, and
displaying the tissue.

25. The method of any one of claims 22-23, further comprising:
imaging a tissue, and
displaying the tissue,
without treating the tissue.

26. The method of any one of claims 22-23, further comprising:
treating a tissue.

27. A method of improving ultrasound imaging alignment by reducing spatial and temporal motion artifacts, comprising:
providing an ultrasonic probe comprising:
an ultrasound imaging transducer adapted for imaging a tissue region,
a motion mechanism attached to the ultrasound imaging transducer;
wherein the ultrasound imaging transducer images a first image with a focal zone sequence order (f₁, . . . , f_{N}), where N>2 when travelling in the first direction,
wherein the ultrasound imaging transducer images a second image with a second focal zone sequence order (f₁, . . . , f_{N}) when travelling in the second direction;
acquiring the first imaging frame;
acquiring the second imaging frame;
calculating offsets between the first imaging frame and the second imaging frame to determine a lateral misregistration;
displaying the first imaging frame; and
displaying the second imaging frame with the offsets applied to reduce a temporal motion artifact.

28. The method of claim 27, further comprising:
calculating an optimized image with at least one trigger offset; and
applying the at least one trigger offset to subsequent image acquisitions,
wherein the lateral misregistration is reduced due to application of the at least one trigger offset.

29. A method of improving ultrasound imaging alignment by reducing spatial and temporal motion artifacts, comprising:
providing an ultrasonic probe comprising:
an ultrasound imaging transducer adapted for imaging a tissue region,
a motion mechanism attached to the ultrasound imaging transducer;
wherein the ultrasound imaging transducer images a first image with a focal zone sequence order (f₁, . . . , f_{N}), where N>2 when travelling in the first direction,
wherein the ultrasound imaging transducer images a second image with a second focal zone sequence order (f₁, . . . , f_{N}) when travelling in the second direction;
acquiring multiple (N>1) imaging frames;
calculating a temporal average of at least two imaging frames;
displaying the temporal average of the at least two imaging frames to reduce a temporal motion artifact.

30. The method of claim 29, further comprising:
calculating an optimized image with at least one trigger offset; and
applying the at least one trigger offset to subsequent image acquisitions,
wherein the averaging of N>1 successive imaging frames is enabled when spatial misregistration between the current and a previously acquired imaging frame is less than a predetermined threshold.

31. A method of improving ultrasound imaging alignment by reducing spatial and temporal motion artifacts, comprising:
providing an ultrasonic probe comprising:
an ultrasound imaging transducer adapted for imaging a tissue region,
a motion mechanism attached to the ultrasound imaging transducer;
wherein the ultrasound imaging transducer images a first image with a focal zone sequence order (f₁, . . . , f_{N}), where N>2 when travelling in the first direction,
wherein the ultrasound imaging transducer images a second image with a second focal zone sequence order (f₁, . . . , f_{N}) when travelling in the second direction;
acquiring the first imaging frame;
acquiring the second imaging frame;
calculating offsets between the first imaging frame and the second imaging frame to determine lateral misregistrations;
calculating a temporal average to the first imaging frame and the second imaging frame;
displaying the temporal average of the first imaging frame and the offset to the second imaging frame to reduce a spatial and temporal motion artifact.

32. The method of claim 31, further comprising:
calculating an optimized image with at least one trigger offset; and
applying the at least one trigger offset to the optimized image,
wherein the lateral misregistration is reduced due to the application of the at least one trigger offset.

33. The method of any one of claims 27-32, further comprising:
imaging a tissue, and
displaying the tissue.

34. The method of any one of claims 27-32, further comprising:
imaging a tissue, and
displaying the tissue,
without treating the tissue.

35. The method of any one of claims 27-32, further comprising treating a tissue.

36. An ultrasound imaging system configured for reducing imaging misalignment, comprising:
an ultrasonic probe comprising an ultrasound therapy transducer adapted to apply ultrasonic therapy to tissue, an ultrasound imaging transducer adapted for imaging the tissue, and a motion mechanism for moving the ultrasound imaging transducer in a first direction and a second direction,
wherein the ultrasound imaging transducer is mechanically attached to the motion mechanism,
wherein the first direction is opposite the second direction,
wherein the ultrasound imaging transducer images with a focal zone sequence order (f₁, . . . , f_{N}), where N>1 when travelling in the first direction,
wherein the ultrasound imaging transducer images with a second focal zone sequence order (f₁, . . . , f_{N}) when travelling in the second direction,
wherein a spatial registration between the first direction imaging and the second direction imaging is improved by staggering a triggering location,
wherein the ultrasound imaging system employs a directionally dependent focal zone sequencing (f₁, . . . , f_{N}) and (f1, . . . , f_{N}) on consecutive A-lines; and
a control module coupled to the ultrasonic probe for controlling the ultrasound imaging transducer.

37. The method of claim 36, wherein N=any one of the group consisting of: 2, 4, 6, and 8.

38. The ultrasound imaging system of claim 36, wherein the first direction of motion of the transducer is any one or more of the group consisting of: linear, rotational, and curved; wherein the second direction is the reversed path of the first direction.

39. The ultrasound imaging system of any one of claims 36-38, wherein the ultrasonic treatment is at least one of: a face lift, a brow lift, a chin lift, an eye treatment, a wrinkle reduction, a décolletage improvement, a buttock lift, a scar reduction, a burn treatment, a skin tightening, a blood vessel reduction, a treatment of a sweat gland, a sun spot removal, a fat treatment, a cellulite treatment, a vaginal rejuvenation, an acne treatment, and abdominal laxity treatment.

40. An ultrasound imaging system having one or more of the features described in the foregoing description.

41. A method of reducing imaging misalignment in a moving ultrasound transducer having one or more of the features described in the foregoing description.
